(19) United States
(12) Patent Application Publication
(10) Pub. No.: US 2026/0221256 A1
(43) Pub. Date: Jul. 30, 2026

(54) A METHOD AND A SYSTEM FOR GENERATING TREATMENT REGIMENS FOR A MEDICAL CONDITION FOR A SUBJECT IN NEED THEREOF

(71) Applicants: Agency for Science, Technology and Research, Singapore (SG); Singapore Health Services Pte Ltd, Singapore (SG)

(72) Inventors: Milashini NAMBIAR, Singapore (SG); Pavitra KRISHNASWAMY, Singapore (SG); Feri GURETNO, Singapore (SG); Yu En CHAN, Singapore (SG); Yong Mong BEE, Singapore (SG)

(21) Appl. No.: 19/144,651

(22) PCT Filed: Dec. 29, 2023
(86) PCT No.: PCT/SG2023/050864

§ 371 Date: Jun. 30, 2025

(30) Foreign Application Priority Data
Dec. 30, 2022 (SG) 10202260646Y

(51) Int. Cl. G16H 20/10; G16H 50/20; G16H 50/30; G16H 50/70

(52) U.S. Cl. CPC G16H 20/10; G16H 50/20; G16H 50/30; G16H 50/70

(57) ABSTRACT

There is provided a method and a system for generating treatment regimens for a medical condition for a subject in need thereof, the method comprising, parsing pre-processed data of the subject through an optimizer to obtain a list of feasible treatment regimens; parsing a selected subset of the list of feasible treatment regimens through a trained artificial intelligence (AI) model to obtain predicted clinical outcomes for each of the selected subset of the list of feasible treatment regimens; obtaining an associated cost for each of the selected subset treatment regimens; obtaining an associated preference score for each of the selected subset of treatment regimens; generating an associated value score for each of the selected subset of treatment regimens, said associated value score based on the predicted clinical outcomes, associated costs, and associated preference score of the selected subset of the list of feasible treatment regimens; and generating one or more recommended treatment regimens based on the associated value scores, each recommended treatment regimen comprising a drug mix and dose regimen.

### TECHNICAL FIELD

The present disclosure relates broadly to a method and a system for generating treatment regimens for a medical condition for a subject in need thereof.

### BACKGROUND

The choice of pharmacotherapy in chronic diseases (e.g., diabetes, hypertension, hyperlipidemia) is becoming increasingly complex and challenging. Practitioners are routinely faced with the task of choosing amongst a large number of drugs, while accounting for multifactorial risks and benefits and individual patient needs. Currently, practitioners rely on evidence based clinical guidelines to narrow the treatment choices. However, these guidelines alone do not resolve uncertainties in assessing comparative effectiveness and addressing heterogeneity in responsiveness and tolerance of individual patients to treatment. Thus, there is growing interest in data-driven treatment recommendation tools that can facilitate more individualized treatment choices and enable better outcomes.

Existing treatment recommendation solutions are primarily based on guideline documentation or use electronic medical record (EMR) data to optimize clinical outcome targets. However, these solutions do not resolve the task of generating individualized treatment recommendations that are consistent with both clinical guidelines and multifactorial practice considerations. For example, guideline-based approaches have limited ability to learn and optimize individualized treatment effects. On the other hand, optimization approaches based on EMR data are limited in the ability to account for granular drug name and dose level specifics required for consistency with guidelines and practice preferences. Furthermore, both types of approaches have limited ability to adapt to multifactorial considerations (e.g., cost, side effect or lifestyle change burdens that are difficult to pre-specify or explicitly model) that tend to influence practical adoption at point-of-care. Hence, there is a need for decision support frameworks that can leverage real-world patient data to individualize treatment for optimal outcomes while (a) comprehensively accounting for guidelines and practice needs, and also (b) allowing for dynamic adaptation to multifactorial considerations at the point of care consult.

Thus, there is a need for a method and a system for generating treatment regimens for a medical condition for a subject in need thereof, that seek to address or alleviate at least one of the above problems.

### SUMMARY

In one aspect, there is provided a method of generating treatment regimens for a medical condition for a subject in need thereof, the method comprising, parsing pre-processed data of the subject through an optimizer to obtain a list of feasible treatment regimens; parsing a selected subset of the list of feasible treatment regimens through a trained artificial intelligence (AI) model to obtain predicted clinical outcomes for each of the selected subset of the list of feasible treatment regimens; obtaining an associated cost for each of the selected subset of treatment regimens; obtaining an associated preference score for each of the selected subset of treatment regimens; generating an associated value score for each of the selected subset of treatment regimens, said associated value score based on the predicted clinical outcomes, associated costs, and associated preference scores of the selected subset of the list of feasible treatment regimens; and generating one or more recommended treatment regimens based on the associated value scores, each recommended treatment regimen comprising a drug mix and dose regimen.

In one embodiment of the method, generating an associated value score for each of the selected subset of treatment regimens comprises, calculating a predicted percentage improvement in clinical outcome for the selected subset of treatment regimens, calculating a weighted percentage reduction in associated cost, and calculating a weighted version of the associated preference score, of the selected subset of treatment regimens.

In one embodiment of the method, generating an associated value score for each of the selected subset of treatment regimens is based on an optimization of the following value function:

$$value ( Rec ) = \frac{{H}_{c} - PRED ( P , Rec )}{{H}_{c}} + {w}_{o} \frac{COST ( {Rec}_{curr} ) - COST ( Rec )}{COST ( {Rec}_{curr} )} + {w}_{1} PREF ( P , Rec )$$

wherein,
P is a patient visit profile,
Rec_{curr} is a current treatment regimen,
Rec is a recommended treatment regimen,
H_{c} is a pre-visit measurement of the clinical outcome,
PRED is a clinical outcome prediction function of the trained AI model which outputs a predicted clinical outcome based on an input treatment regimen,
PREF is a preference scoring function which outputs a score of an input treatment regimen,
COST is a cost function which outputs a cost of an input treatment regimen,
w₀ is a hyperparameter which controls a trade-off between improvement in clinical outcome and cost,
w₁ is a hyperparameter which controls a trade-off between improvement in clinical outcome and a preferred medication prescription.

In one embodiment of the method, generating one or more recommended treatment regimens is based on obtaining one or more treatment regimens that maximize the value function.

In one embodiment of the method, parsing the pre-processed data of the subject through an optimizer to obtain the list of feasible treatment regimens comprises accessing a clinical knowledge database to obtain the list of feasible treatment regimens.

In one embodiment of the method, parsing a selected subset of the list of feasible treatment regimens through a trained artificial intelligence (AI) model comprises, selecting, from the list of feasible treatment regimens, a first list of feasible treatment regimens comprising a first medication; sorting the first list of feasible treatment regimens based on a dosage quantity of the first medication to obtain a first ordered list of feasible doses of the first medication; obtaining a first ordered list of treatment regimens corresponding with the first ordered list of feasible doses of the first medication; parsing the first ordered list of treatment regimens to obtain an inflection treatment regimen associated with an inflection point of the value function, using a first binary search algorithm, and parsing the first ordered list of treatment regimens between the inflection treatment regimen and a maximum treatment regimen, using a second binary search algorithm, to obtain one or more recommended treatment regimens each having an associated value score that optimizes the value function.

In one embodiment of the method, parsing a selected subset of the list of feasible treatment regimens through a trained artificial intelligence (AI) model comprises, selecting, from the list of feasible treatment regimens, a second list of feasible treatment regimens that excludes the first medication; sorting the second list of feasible treatment regimens based on a dosage quantity of a second medication to obtain a second ordered list of feasible doses of the second medication; obtaining a second ordered list of treatment regimens corresponding with the second ordered list of feasible doses of the second medication; parsing the second ordered list of treatment regimens to obtain an inflection treatment regimen associated with an inflection point of the value function, using the first binary search algorithm, and parsing the second ordered list of treatment regimens between the inflection treatment regimen and a maximum treatment regimen of the second medication, using the second binary search algorithm, to obtain one or more recommended treatment regimens each having an associated value score that optimizes the value function.

In one embodiment of the method, parsing a selected subset of the list of feasible treatment regimens through a trained artificial intelligence (AI) model comprises, selecting, from the list of feasible treatment regimens, a third list of feasible treatment regimens comprising a specific dosage of the first medication; sorting the third list of feasible treatment regimens based on a dosage quantity of a third medication to obtain a third ordered list of feasible doses of the third medication; obtaining a third ordered list of treatment regimens corresponding with the third ordered list of feasible doses of the third medication; parsing the third ordered list of treatment regimens to obtain an inflection treatment regimen associated with an inflection point of the value function, using the first binary search algorithm, and parsing the third ordered list of treatment regimens between the inflection treatment regimen and a maximum treatment regimen, using the second binary search algorithm, to obtain one or more recommended treatment regimens each having an associated value score that optimizes the value function.

In one embodiment of the method, the associated preference score for each of the selected subset of treatment regimens is based on one or more of: indicator functions of whether a prioritized medication is prescribed; linear functions in the dose of each medication; and indicator functions of whether each medication is prescribed.

In one embodiment of the method, the method further comprises pre-processing data of the subject by a data pre-processor, said pre-processing step comprising, extracting one or more drug mixes and dose regimens from an electronic medical record (EMR) of the subject; computing a first vector representing a drug mix and dose regimen of a first visit treatment regimen, wherein the first vector has a length equal to the number of generic name drugs for the medical condition, and each entry in the first vector represents a periodic dose of a corresponding generic name drug; and computing a second vector representing a drug mix and dose regimen of a second visit treatment regimen, wherein the second vector has the same length as the first vector, and each entry in the second vector represents the periodic dose of a corresponding generic name drug at the second visit.

In one embodiment of the method, the method further comprises performing a plurality of validation checks on the data of the subject to be pre-processed; and triggering an alert message when a failure in one of the validation checks is detected.

In one embodiment of the method, the method further comprises training the AI model, said training comprising, pre-processing training data comprising retrospective data from electronic medical records of a group of patients having the medical condition; the electronic medical records comprising the patients' medical profile data, historical drug mixes and dose regimen data and associated clinical outcome for each historical drug mix and dose regimen; feeding the pre-processed training data into a machine learning algorithm to model clinical outcomes under different drug mixes and dose regimens; and obtaining the trained AI model that is configured to predict clinical outcomes using the subject's medical profile and a treatment regimen as input.

In one embodiment of the method, the method further comprises displaying a first and second recommended treatment regimens, predicted clinical outcomes and associated costs via an interactive dashboard; wherein the first recommended treatment regimen is generated based on an optimization of the value function with the hyperparameter w₀ set to zero; and wherein the second recommended treatment regimen is generated based on an optimization of the value function with the hyperparameter w₀ set to a positive value.

In one embodiment of the method, the method further comprises regenerating the second recommended treatment regimen displayed on the interactive dashboard in response to user input to generate a lower-cost option, said regenerating step comprising, increasing the value of the hyperparameter w₀; generating an associated value score for each of the selected subset of treatment regimens by optimizing the value function based on the increased value of the hyperparameter w₀; generating a lower-cost treatment regimen based on the associated value score; and presenting the lower-cost treatment regimen as the regenerated second recommended treatment regimen; wherein the regeneration step is configured to be triggered iteratively by the user until the lowest-cost option is presented as the regenerated second recommended treatment regimen.

In one embodiment of the method, the method further comprises simulating clinical outcomes of one or more customized treatment regimens based on manually entered user input, said simulation step comprising, preprocessing the one or more customized treatment regimens; parsing the one or more customized treatment regimens through the trained AI model to obtain predicted clinical outcomes for the one or more customized treatment regimens; and presenting the predicted clinical outcomes for the one or more customized treatment regimens via the interactive dashboard.

In another aspect, there is provided a system for generating treatment regimens for a medical condition for a subject in need thereof, the system comprising, an optimizer for parsing pre-processed data of the subject to obtain a list of feasible treatment regimens; and a trained AI model for parsing a selected subset of the list of feasible treatment regimens to obtain predicted clinical outcomes for each of the selected subset of the list of feasible treatment regimens; wherein the optimizer is further configured to: obtain an associated cost for each of the selected subset of treatment regimens; obtain an associated preference score for each of the selected subset of treatment regimens; generate an associated value score for each of the selected subset of treatment regimens, said associated value score based on the predicted clinical outcomes, associated costs, and associated preference scores of the selected subset of the list of feasible treatment regimens; and generate one or more recommended treatment regimens based on the associated value scores, each recommended treatment regimen comprising a drug mix and dose regimen.

In one embodiment of the system, the optimizer is configured to generate an associated value score for each of the selected subset of treatment regimens by calculating a predicted percentage improvement in clinical outcome for the selected subset of treatment regimens, calculating a weighted percentage reduction in associated cost, and calculating a weighted version of the associated preference score of the selected subset of treatment regimens.

In one embodiment of the system, the optimizer is configured to generate an associated value score for each of the selected subset of treatment regimens based on an optimization of the following value function:

$$value ( Rec ) = \frac{{H}_{c} - PRED ( P , Rec )}{{H}_{c}} + {w}_{o} \frac{COST ( {Rec}_{curr} ) - COST ( Rec )}{COST ( {Rec}_{curr} )} + {w}_{1} PREF ( P , Rec )$$

wherein,
P is a patient visit profile,
Rec_{curr} is a current treatment regimen,
Rec is a recommended treatment regimen,
H_{c} is a pre-visit measurement of the clinical outcome,
PRED is a clinical outcome prediction function of the trained AI model which outputs a predicted clinical outcome based on an input treatment regimen,
PREF is a preference scoring function which outputs a score of an input treatment regimen,
COST is a cost function which outputs a cost of an input treatment regimen,
w₀ is a hyperparameter which controls a trade-off between improvement in clinical outcome and cost,
w₁ is a hyperparameter which controls a trade-off between improvement in clinical outcome and a preferred medication prescription.

In one embodiment of the system, the optimizer is configured to generate one or more recommended treatment regimens that maximize the value function.

In one embodiment of the system, the system further comprises a clinical knowledge database coupled to the optimizer, wherein the optimizer is configured to access the clinical knowledge database to obtain the list of feasible treatment regimens.

In one embodiment of the system, the optimizer is configured to cooperate with the trained AI model to generate the one or more recommended treatment regimens by, selecting, from the list of feasible treatment regimens, a first list of feasible treatment regimens comprising a first medication; sorting the first list of feasible treatment regimens based on a dosage quantity of the first medication to obtain a first ordered list of feasible doses of the first medication; obtaining a first ordered list of treatment regimens corresponding with the first ordered list of feasible doses of the first medication; parsing the first ordered list of treatment regimens to obtain an inflection treatment regimen associated with an inflection point of the value function, using a first binary search algorithm, and parsing the first ordered list of treatment regimens between the inflection treatment regimen and a maximum treatment regimen, using a second binary search algorithm, to obtain one or more recommended treatment regimens each having an associated value score that optimizes the value function.

In one embodiment of the system, the optimizer is further configured to cooperate with the trained AI model to generate the one or more recommended treatment regimens by, selecting, from the list of feasible treatment regimens, a second list of feasible treatment regimens that excludes the first medication; sorting the second list of feasible treatment regimens based on a dosage quantity of a second medication to obtain a second ordered list of feasible doses of the second medication; obtaining a second ordered list of treatment regimens corresponding with the second ordered list of feasible doses of the second medication; parsing the second ordered list of treatment regimens to obtain an inflection treatment regimen associated with an inflection point of the value function, using the first binary search algorithm, and parsing the second ordered list of treatment regimens between the inflection treatment regimen and a maximum treatment regimen of the second medication, using the second binary search algorithm, to obtain one or more recommended treatment regimens each having an associated value score that optimizes the value function.

In one embodiment of the system, the optimizer is further configured to cooperate with the trained AI model to generate the one or more recommended treatment regimens by, selecting, from the list of feasible treatment regimens, a third list of feasible treatment regimens comprising a specific dosage of the first medication; sorting the third list of feasible treatment regimens based on a dosage quantity of a third medication to obtain a third ordered list of feasible doses of the third medication; obtaining a third ordered list of treatment regimens corresponding with the third ordered list of feasible doses of the third medication; parsing the third ordered list of treatment regimens to obtain an inflection treatment regimen associated with an inflection point of the value function, using the first binary search algorithm, and parsing the third ordered list of treatment regimens between the inflection treatment regimen and a maximum treatment regimen, using the second binary search algorithm, to obtain one or more recommended treatment regimens each having an associated value score that optimizes the value function.

In one embodiment of the system, the associated preference score for each of the selected subset of treatment regimens is based on one or more of: indicator functions of whether a prioritized medication is prescribed; linear functions in the dose of each medication; and indicator functions of whether each medication is prescribed.

In one embodiment of the system, the system further comprises a data pre-processor configured to extract one or more drug mixes and dose regimens from an EMR of the subject; compute a first vector representing a drug mix and dose regimen of a first visit treatment regimen, wherein the first vector has a length equal to the number of generic name drugs for the medical condition, and each entry in the first vector represents a periodic dose of a corresponding generic name drug; and compute a second vector representing a drug mix and dose regimen of a second visit treatment regimen, wherein the second vector has the same length as the first vector, and each entry in the second vector represents the periodic dose of a corresponding generic name drug at the second visit treatment regimen.

In one embodiment of the system, the system further comprises an input validation processor configured to perform a plurality of validation checks on the data of the subject to be pre-processed; and trigger an alert message when a failure in one of the validation checks is detected.

In one embodiment of the system, the AI model is configured to predict the clinical outcome using the subject's medical profile and a treatment regimen as input; and wherein the AI model is trained by, pre-processing training data comprising retrospective data from electronic medical records of a group of patients having the medical condition; the electronic medical records comprising the patients' medical profile data, historical drug mixes and dose regimen data and associated clinical outcome for each historical drug mix and dose regimen; and feeding the pre-processed training data into a machine learning algorithm to model clinical outcomes under different drug mixes and dose regimens.

In one embodiment of the system, the system further comprises an interactive dashboard configured to, display a first and second recommended treatment regimens, predicted clinical outcomes and associated costs; wherein the first recommended treatment regimen is generated based on an optimization of the value function with the hyperparameter w₀ set to zero; and wherein the second recommended treatment regimen is generated based on an optimization of the value function with the hyperparameter w₀ set to a positive value.

In one embodiment of the system, the optimizer is further configured to regenerate the second recommended treatment regimen in response to user input to generate a lower-cost option for display on the interactive dashboard; and wherein the optimizer is configured to iteratively regenerate the second recommended treatment regimen in response to user input such that with each iteration, a lower-cost option is generated for display on the interactive dashboard until the lowest-cost option is presented as the regenerated second recommended treatment regimen.

In one embodiment of the system, the system is further configured to simulate clinical outcomes of one or more customized treatment regimens based on manually entered user input; wherein the data pre-processor is configured to preprocess the one or more customized treatment regimens; the trained AI model is configured to parse the one or more customized treatment regimens to obtain predicted clinical outcomes for the one or more customized treatment regimens; and the interactive dashboard is configured to present the predicted clinical outcomes for the one or more customized treatment regimens.

In another aspect, there is provided a non-transitory computer readable storage medium having stored thereon instructions for instructing a processing unit of an apparatus to execute a method of generating treatment regimens for a medical condition for a subject in need thereof, the method comprising, parsing pre-processed data of the subject through an optimizer to obtain a list of feasible treatment regimens; parsing a selected subset of the list of feasible treatment regimens through a trained artificial intelligence (AI) model to obtain predicted clinical outcomes for each of the selected subset of the list of feasible treatment regimens; obtaining an associated cost for each of the selected subset treatment regimens; obtaining an associated preference score for each of the selected subset of treatment regimens; generating an associated value score for each of the selected subset of treatment regimens, said associated value score based on the predicted clinical outcomes, associated costs, and associated preference score of the selected subset of the list of feasible treatment regimens; and generating one or more recommended treatment regimens based on the associated value scores, each recommended treatment regimen comprising a drug mix and dose regimen.

### Definitions

The term “clinical outcome” as used herein is to be interpreted broadly to refer to a result of impact of a medical treatment on a patient's health. The term “clinical outcome” represents measurable changes in health status, symptoms, or overall condition of an individual that are attributed to a specific healthcare intervention or therapeutic approach. One approach of assessing a clinical outcome associated with a treatment of a medical condition is by measuring one or more clinical markers associated with the medical condition.

The term “clinical marker” as used herein is to be interpreted broadly to refer to a measurable and quantifiable characteristic or indicator that is used to assess a particular aspect of a patient's health. Clinical markers may be used in the diagnosis, monitoring, and management of various medical conditions. Clinical markers may include physical signs, laboratory test results, imaging findings, or other objective measures that provide information about the presence or progression of a disease, the effectiveness of a treatment, or the overall health status of an individual. Examples of clinical markers include but are not limited to blood glucose levels, blood pressure, cholesterol levels, C-reactive Protein (CRP), Hemoglobin A1c (HbA1c), liver enzymes, and white blood cell count.

The term “dose regimen” as used herein refers to a prescribed schedule and specific instructions for the administration of a medication or drug. A dose regimen includes details such as dosage amount, frequency of administration, and duration of treatment. The goal of a dose regimen is to ensure that a patient receives the right amount of medication at the right intervals to achieve the desired therapeutic effect while minimizing the risk of side effects or complications. The term “dosage amount” refers to the quantity of the medication to be taken at each administration. Dosage amount may be expressed in terms of milligrams, micrograms, milliliters, etc., depending on the form of the medication (e.g., tablets, capsules, liquid). The term “frequency of administration” refers to the number of times the medication should be taken per day or per week. Common frequencies include once daily, twice daily, every 8 hours, etc. The term “duration of treatment” refers to the length of time the medication should be taken. Some medications are prescribed for a short duration (e.g., for a week), while others may be used on a long-term basis.

The term “effective amount” as used herein is to be interpreted as an amount that is sufficient to carry out its intended effect. For example, when an “effective amount” is used to refer to the administration of a compound, it can refer to the situation where the compound is administered at a dosage and/or for a period of time necessary to achieve the desired result.

The term “subject” as used herein refers to an individual. The term “subject” as used herein may refer to a human patient. A subject having a medical condition refers to a subject e.g., patient that can be confirmed to have the disease (e.g., Type 2 diabetes) based on a set of signs, symptoms, tests and criteria used by a person skilled in the art (e.g., medical professional) to diagnose the disease.

The term “treating” as used herein is to be interpreted to mean alleviating, ameliorating, or modulating a disease/disorder/medical condition, or one or more clinical symptoms indicative of the disease/disorder/medical condition. For example, where the medical condition is diabetes, alleviation, amelioration, or modulation of a clinical symptom may include a decrease or stabilization in blood glucose levels in a treated subject compared to pretreatment blood glucose levels. The term “treating” as used herein is also to be interpreted to mean preventing or delaying the onset or development or progression of a disease/disorder/medical condition. As used herein, a subject is “in need of” a treatment if the subject would benefit biologically, medically or in quality of life from such treatment. The disease may not need to be effectively treated eventually.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

The description herein may be, in certain portions, explicitly or implicitly described as algorithms and/or functional operations that operate on data within a computer memory or an electronic circuit. These algorithmic descriptions and/or functional operations are usually used by those skilled in the information/data processing arts for efficient description. An algorithm is generally relating to a self-consistent sequence of steps leading to a desired result. The algorithmic steps can include physical manipulations of physical quantities, such as electrical, magnetic or optical signals capable of being stored, transmitted, transferred, combined, compared, and otherwise manipulated.

Further, unless specifically stated otherwise, and would ordinarily be apparent from the following, a person skilled in the art will appreciate that throughout the present specification, discussions utilizing terms such as “scanning”, “calculating”, “determining”, “replacing”, “generating”, “initializing”, “outputting”, and the like, refer to action and processes of an instructing processor/computer system, or similar electronic circuit/device/component, that manipulates/processes and transforms data represented as physical quantities within the described system into other data similarly represented as physical quantities within the system or other information storage, transmission or display devices etc.

The description also discloses relevant device/apparatus for performing the steps of the described methods. Such apparatus may be specifically constructed for the purposes of the methods, or may comprise a general purpose computer/processor or other device selectively activated or reconfigured by a computer program stored in a storage member. The algorithms and displays described herein are not inherently related to any particular computer or other apparatus. It is understood that general purpose devices/machines may be used in accordance with the teachings herein. Alternatively, the construction of a specialized device/apparatus to perform the method steps may be desired.

In addition, it is submitted that the description also implicitly covers a computer program, in that it would be clear that the steps of the methods described herein may be put into effect by computer code. It will be appreciated that a large variety of programming languages and coding can be used to implement the teachings of the description herein. Moreover, the computer program if applicable is not limited to any particular control flow and can use different control flows without departing from the scope of the invention.

Furthermore, one or more of the steps of the computer program if applicable may be performed in parallel and/or sequentially. Such a computer program if applicable may be stored on any computer readable medium. The computer readable medium may include storage devices such as magnetic or optical disks, memory chips, or other storage devices suitable for interfacing with a suitable reader/general purpose computer. In such instances, the computer readable storage medium is non-transitory. Such storage medium also covers all computer-readable media e.g., medium that stores data only for short periods of time and/or only in the presence of power, such as register memory, processor cache and Random Access Memory (RAM) and the like. The computer readable medium may even include a wired medium such as exemplified in the Internet system, or wireless medium such as exemplified in Bluetooth technology. The computer program when loaded and executed on a suitable reader effectively results in an apparatus that can implement the steps of the described methods.

The example embodiments may also be implemented as hardware modules. A module is a functional hardware unit designed for use with other components or modules. For example, a module may be implemented using digital or discrete electronic components, or it can form a portion of an entire electronic circuit such as an Application Specific Integrated Circuit (ASIC). A person skilled in the art will understand that the example embodiments can also be implemented as a combination of hardware and software modules.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a method and a system for generating treatment regimens for a medical condition for a subject in need thereof are disclosed hereinafter.

### Method of Generating Treatment Regimens for a Medical Condition

In various embodiments, there is provided a method of generating treatment regimens for a medical condition for a subject in need thereof, the method comprising, parsing pre-processed data of the subject through an optimizer to obtain a list of feasible treatment regimens; parsing a selected subset of the list of treatment regimens through a trained artificial intelligence (AI) model to obtain predicted clinical outcomes for each of the selected subset of the list of feasible treatment regimens; obtaining an associated cost for each of the selected subset of treatment regimens; obtaining an associated preference score for each of the selected subset of treatment regimens; generating an associated value score for each of the selected subset of treatment regimens, said associated value score based on the predicted clinical outcomes, associated costs and associated preference scores of the selected subset of the list of feasible treatment regimens; and generating one or more recommended treatment regimens based on the associated value scores, each recommended treatment regimen comprising a drug mix and dose regimen. In various embodiments, the method is a computer-implemented method.

In various embodiments, the treatment regimen, e.g., recommended treatment regimen, may comprise or may consist of a drug/medication mix and a dose regimen. In various embodiments, the drug mix may comprise one or more drugs/medications for treating the medical condition. In various embodiments, the terms “drug” and “medication” may be used interchangeably. For example, the drug mix may comprise one drug only, two drugs, three drugs, or more than three drugs. In various embodiments, where the drug mix comprises two or more drugs, the drugs may be different from each other, i.e., having different generic names. In various embodiments, the dose regimen of each drug in the drug mix may be expressed as a total dosage amount of the drug to be administered per day, e.g., grams per day or milligrams per day.

In various embodiments, the drug in the drug mix of the treatment regimen may be represented by its generic name. That is, the drug may be represented in terms of its generic name (i.e., chemical name or non-proprietary name), as opposed to being represented in terms of its brand or trade name (i.e., a more specific level of naming). In various embodiments, representing a drug in a treatment regimen in terms of its generic name may advantageously provide a standardized and universally recognized way of referring to the drug. Unlike a brand or trade name which can vary across countries and regions, generic names are consistent and allow for uniform representation in a machine learning model. In various embodiments, representing a drug in a treatment regimen in terms of its generic name may enable the presently disclosed method and associated system to tune the dosage of the drug in the treatment regimen. A uniform representation of drugs may advantageously ensure accurate and consistent dosage information when tuning dosage regimens.

In various embodiments, the presently disclosed approach of representing medications at the generic name level, as compared to existing approaches of representing medications at the drug class/subgroup level or as categorical combinations of drug classes, may further advantageously provide a higher level of granularity and allow the presently disclosed approach to extend beyond choice of broad drug subgroups or classes and to also specifically account for the practically relevant dimensions of drug name and dose.

In various embodiments, the medical condition may be a chronic medical condition. In various embodiments, the medical condition may be treatable by administration of medication, e.g., on a long-term basis. In various embodiments, the medical condition may include but is not limited to diabetes mellitus, e.g., type 2 diabetes, hypertension (high blood pressure), hyperlipidemia (high cholesterol), epilepsy, and rheumatoid arthritis. In various embodiments, the predicted clinical outcome may be assessed using one or more clinical markers associated with the medical condition. For example, where the medical condition is type 2 diabetes, the clinical marker, glycated hemoglobin HbA1c, may be used for assessing treatment effectiveness and guiding adjustments in treatment regimen to improve clinical outcomes for individuals with type 2 diabetes. It will be appreciated that the predicted clinical outcomes of other medical conditions may be measured using suitable clinical markers known to a person skilled in the art.

In various embodiments, the optimizer may be configured to perform multi-objective optimization based on a plurality of inputs (e.g., preprocessed data of the subject, predicted clinical outcome associated with the medical condition, and clinical guidelines data) to generate the one or more treatment recommendations. In various embodiments, the optimizer may be configured to generate the one or more recommended treatment regimens by selecting one or more treatment regimens from the selected subset of the list of treatment regimens with associated values scores that are optimized for the subject. In various embodiments, an associated value score for a treatment regimen from the selected subset may be generated by calculating a predicted percentage improvement in clinical outcome for the treatment regimen from the selected subset, calculating a weighted percentage reduction in associated cost, and calculating a weighted version of the associated preference score, of the treatment regimen from the selected subset.

In various embodiments, the recommended treatment regimen may comprise or may consist of a drug mix and dose regimen (i.e., a medication recommendation). In various embodiments, the recommended treatment regimen may be accompanied by additional/supplementary information, including but not limited to estimates of cost, predicted clinical outcome, confidence or uncertainty level of the predicted clinical outcome. It will be appreciated that such additional information is not the recommended treatment regimen per se but may be useful in facilitating an informed decision-making process by end-users whether to adopt the recommended treatment regimen. In various embodiments, the recommended treatment regimen may be accompanied by a cost of the drug mix (e.g., expressed as cost per week, month, or year). In various embodiments, the recommended treatment regimen may be accompanied by a predicted clinical outcome for the medical condition for a specific time horizon (e.g., prediction data of a clinical marker such as HbA1c, 6 months from current visit). In various embodiments, the recommended treatment regimen may be accompanied by an uncertainty estimate of the clinical outcome if the drug mix were to be administered for a set period of time. In various embodiments, the one or more recommended treatment regimens may include at least a first treatment recommendation representing a minimum-intensity treatment regimen biased towards achieving or maintaining control of the clinical marker at a desired level, and a second treatment recommendation biased towards achieving a lower-cost treatment regimen than the first treatment recommendation.

In various embodiments therefore, the presently disclosed method is advantageously capable of performing multi-objective optimization to generate and compare a plurality of treatment regimens that not only optimizes target clinical outcomes (e.g., HbA1c reduction), but also considers different trade-offs such as associated costs of treatment regimens and practicability of treatment regimens.

### Data Pre-Processing

In various embodiments, the method may further comprise pre-processing data of the subject by a data pre-processor. In various embodiments, pre-processing data of the subject may comprise extracting one or more drug mixes and dose regimens or dose representations from an electronic medical record (EMR) of the subject. In various embodiments, the data of the subject may be comprised in an EMR database. In various embodiments, the EMR database may be a centralized database. In various embodiments, the EMR database may include records of the subject, including but not limited to diagnoses, prescription history and laboratory test results. For example, the EMR database may include the subject's medical profile data, historical drug mixes and dose regimen data and associated clinical outcome for each historical drug mix and dose regimen. In various embodiments, the diagnoses, prescription history and laboratory test results of the subject may be recorded at multiple healthcare sites. In various embodiments, a healthcare site may include a hospital, specialty center, or primary care site (e.g., clinics) where a patient has either received treatment for the medical condition or a laboratory test for a clinical marker, e.g., blood glucose. Advantageously, the use of a centralized EMR database by the presently disclosed method facilitates access to the records of the subject, regardless of healthcare sites.

In various embodiments, the pre-processing of data functions to condense EMR data into representations of the subject profile and prescription history at each visit to a healthcare site. In general, EMR data may be complex and may contain free text that is not compatible with algorithms, missing data and/or errors. In various embodiments therefore, the data pre-processing routine advantageously helps to address these issues and convert the EMR data into a form that is usable by a system implementing the presently disclosed method.

In various embodiments, pre-processing data of the subject may further comprise, constructing one or more configuration files based on historical EMR data to facilitate pre-processing of data. In various embodiments, the one or more configuration files may facilitate subsequent computation of vectors representing drug mixes and dose regimens. In various embodiments, the configuration files are configured to map values on complex drug name information to generic name information. In various embodiments, the configuration files are configured to map drug frequency information (typically listed in free text, using abbreviations of Latin terms) to numerical information. In various embodiments, the configuration files are configured to store typical doses (e.g., median, minimum, maximum) of different generic name medications.

In various embodiments, pre-processing data of the subject may further comprise determining whether a treatment regimen of the subject, if any, is active. In various embodiments, determining whether a treatment regimen of the subject is active may comprise determining, with respect to the subject's current visit date, previous treatment regimen(s) that are active on the subject by filtering out previous treatment regimen(s) with prescription window(s) that do not encompass the current visit date. In various embodiments, the term “prescription window” as used herein with respect to a treatment regimen refers to a time period or duration calculated from a prescribed date of a treatment regimen to the prescribed date of the treatment regimen plus a prescription duration and a grace period.

In various embodiments, pre-processing data of the subject may further comprise pre-processing treatment regimens. In various embodiments, pre-processing treatment regimens may comprise one or more of the following: mapping complex name drug information to generic name drug information, mapping text-based drug frequency information to numerical drug frequency information, and computing periodic dose, e.g., daily dose, for each generic name drug. In various embodiments, pre-processing treatment regimens may further comprise checking and, if applicable, changing a unit of measure (e.g., milligrams, grams) of the drug information such that the dose obtained has the correct order of magnitude.

In various embodiments, pre-processing data of the subject may further comprise computing a first vector representing a drug mix and dose regimen of a first visit treatment regimen, e.g., previous visit treatment regimen. In various embodiments, the first vector has a length equal to the number of generic name drugs for the medical condition, and each entry in the first vector represents a periodic dose of a corresponding generic name drug. In various embodiments, pre-processing data of the subject may further comprise computing a second vector representing a drug mix and dose regimen of a second visit treatment regimen, e.g., current visit treatment regimen. In various embodiments, the second vector has the same length as the first vector, and each entry in the second vector represents the periodic dose of a corresponding generic name drug in the second visit treatment regimen. In various embodiments, the first and second vectors may each be represented as a list of numeric values, each numeric value representing the periodic dose of a corresponding generic name drug. In various embodiments, the generic name drugs in the first and second vectors have the same order of arrangement. In various embodiments, the numeric value representing the periodic dose of a corresponding generic name drug is a positive value if the treatment regimen includes the corresponding generic name drug. In various embodiments, the numeric value representing the periodic dose of a corresponding generic name drug is zero if the treatment regimen does not include the corresponding generic name drug.

In various embodiments, the first visit treatment regimen may be a previous visit treatment regimen. In various embodiments, the second visit may be a current visit treatment regimen. The current visit treatment regimen may refer to the selected subset of the list of treatment regimens or the recommended treatment regimen generated by the presently disclosed method. In various embodiments, the number of generic name drugs for the medical condition may be obtained from a formulary, which is a list of prescription drugs, medical devices, and treatments approved for use within a particular healthcare system. In various embodiments, the periodic dose may be a daily dose or weekly dose.

### Input Validation Checks

In various embodiments, the method may further comprise performing a plurality of validation checks on the data of the subject to be pre-processed. In various embodiments, the validation checks advantageously ensure that the essential data fields are valid for downstream inference and recommendation generation.

In various embodiments, the validation checks may include checking for compulsory fields and other fields. In various embodiments, compulsory fields may include but are not limited to demographic data such as age, gender, and laboratory results such as creatinine serum, HbA_{1c} laboratory results and validity periods of laboratory results.

In various embodiments, the validation checks for other fields may include checking for previous prescription information. In various embodiments, checking for previous prescription information may include checking whether any previous prescriptions were extracted. In various embodiments, checking for previous prescription information includes checking whether a previous prescription has exceeded a validity period, e.g., validity period of 1 year. In various embodiments, checking for previous prescription information includes checking whether a drug in a previous prescription information has been phased out or is a newly released drug. In various embodiments, checking for previous prescription information may include checking whether dosage information in a previous prescription information is valid (available and correct). In various embodiments, checking for previous prescription information may include checking whether a drug mix in a previous prescription information is contraindicated.

In various embodiments, the method may further comprise triggering an alert message when a failure in one of the validation checks is detected. This allows a user, e.g., clinicians to be notified of the specific issue(s) so that clinicians can check on the input data at their end or proceed to make their own treatment recommendations to the patient. In various embodiments therefore, the input validation advantageously ensures validity of inputs and enhances system safety and robustness.

### Clinical Knowledge Base

In various embodiments, parsing the pre-processed data of the subject through an optimizer to obtain the list of feasible treatment regimens may comprise accessing a clinical knowledge database to obtain the list of feasible treatment regimens from a broader list of potential treatment regimens. In various embodiments, the optimizer may utilize the clinical knowledge base/database to facilitate the search for one or more recommended treatment regimens from the selected subset of the list of feasible treatment regimens. A description of potential treatment regimens and feasible treatment regimens can be found under the “Drug mix and dose optimizer” section.

In various embodiments, the clinical knowledge base refers to a repository of codified guidelines that the optimizer can use to search for one or more recommended treatment regimens from the selected subset of the list of treatment regimens. In various embodiments, the clinical knowledge base may comprise a broad list/menu of potential treatment regimens, from which the list of feasible treatment regimens may be obtained. In various embodiments, the clinical knowledge base may comprise rules that act as filters, e.g., for filtering out infeasible treatment regimens, thereby filtering the broad list down to the list of feasible treatment regimens. For example, clinical knowledge base may comprise codified guidelines that take into consideration factors such as contradictions and other preferences, such as not using intensive control for elderly patients or patients with a history of certain medical conditions.

In various embodiments, the clinical knowledge base is a separate/distinct entity from the optimizer. In various embodiments, the clinical knowledge base is communicatively coupled to the optimizer. In various embodiments, the clinical knowledge base may be a knowledge base that is currently available in the art. For example, a suitable knowledge base available in the art may be directly adopted or may be modified for use by the presently disclosed method and associated system. In various embodiments, the clinical knowledge base may be specially designed and constructed for the purpose of the presently disclosed method and associated system.

In various embodiments, the clinical knowledge base may be constructed from clinical knowledge-based guidelines pertaining to the medical condition, e.g., type 2 diabetes, from a plurality of sources. In various embodiments, sources of guidelines for constructing the clinical knowledge base may include but are not limited to Health Sciences Authority (HSA) (https://www.hsa.gov.sg/), MIMS (https://www.mims.com/singapore/), and relevant clinical specialty bodies. For the use case of diabetes, clinical specialty bodies may include the American Diabetes Association (ADA) (https://www.diabetes.org/), and the European Association for the Study of Diabetes (EASD) (https://www.easd.org/). In various embodiments, sources of guidelines for constructing the clinical knowledge base may also include knowledge from formularies. In various embodiments, a formulary refers to a list of prescription drugs containing each drug's dosage form, strength, route of administration, and any specific requirements for use. In various embodiments, formularies may be created and maintained by various organizations such as healthcare authority of a country, and insurance companies. In various embodiments therefore, the clinical knowledge base may advantageously ensure that the one or more recommended treatment regimens are consistent and/or compliant with (i.e., adheres to) clinical guidelines.

In various embodiments, the clinical knowledge base may comprise a plurality of sets of rules, e.g., decision rules, constructed/codified based on the clinical knowledge-based guidelines obtained from the plurality of sources. In various embodiments, each set may comprise one or more rules. In various embodiments, the plurality of sets of rules derived from human expertise or domain knowledge are represented in a structured format that the optimizer can understand and apply. In various embodiments, the plurality of sets of rules may comprise explicit, predefined instructions or conditions that are usable by the optimizer to make decisions. In various embodiments, the set of rules may comprise if-then rules, each if-then rule consisting of a condition (if) and an associated action or outcome (then). For example, the rule may be in the form “If Subject has Profile X, then do not recommend medication A”. In various embodiments, the plurality of sets of rules may advantageously be used to filter a decision space which is then searched by the optimizer to generate the recommended treatment regimens.

In various embodiments, the plurality of sets of rules may be categorized into a plurality of categories of guidelines, such that each set of rules pertains to a particular category of guidelines. In various embodiments, the clinical knowledge base may comprise a set of rules pertaining to medication contraindications, e.g., rules to drop contraindicated medications, based on the guideline that certain medications should not be prescribed depending on the subject's health condition. In various embodiments, the clinical knowledge base may comprise a set of rules pertaining to avoidance of contraindicated combinations of medications, based on the guideline that certain combinations of medications should not be prescribed together. In various embodiments, the clinical knowledge base may comprise a set of rules pertaining to viable doses for each medication, based on the guideline that the prescribed dose of each medication should belong to a discrete set as determined by formulary availability. In various embodiments, the clinical knowledge base may comprise a set of rules pertaining to renal dosing, based on the guideline that the maximum viable dose of each medication may be limited depending on the subject's kidney function. In various embodiments, the clinical knowledge base may comprise a set of rules pertaining to intensification of treatment recommendation, based on the guideline that an increase in severity level resulting from an intensification of treatment recommendation should be capped. In various embodiments, the treatment regimen intensity may be represented as progressively intensive lines of therapy, as applicable to the condition of interest. In various embodiments, the clinical knowledge base may comprise a set of rules pertaining to prioritization of treatment regimens with relatively lower treatment intensities. In various embodiments, the clinical knowledge base may comprise a set of rules pertaining to prioritization or de-prioritization of certain medications due to health benefits and risk. In various embodiments, the clinical knowledge base may comprise data on medication costs. It will be appreciated that the plurality of sets of rules is not limited to the above guidelines. Additional sets of rules may be included to improve the recommendation process.

### Training the AI Model for Predicting Clinical Outcomes

In various embodiments, the method may further comprise training the AI model. In various embodiments, the AI model may be trained to predict a post-visit level of a clinical marker based on a visit medical profile and a treatment regimen (e.g., treatment regimen from the selected subset of the list of feasible treatment regimens).

In various embodiments, training the AI model may comprise pre-processing training data. In various embodiments, the training data may comprise retrospective data from electronic medical records of a group of patients having the medical condition. In various embodiments, the electronic medical records may comprise the patients' medical profile data, historical treatment regimens comprising drug mixes and dose regimen data and associated clinical outcome for each historical treatment regimen.

In various embodiments, the historical treatment regimens may be pre-processed such that drug mixes and dose regimen data are represented in a vector format. In various embodiments, the visit medical profile representation may be condensed via feature selection, using techniques such as e.g., the Least Absolute Shrinkage and Selection Operator (LASSO).

In various embodiments, training the AI model may further comprise feeding the pre-processed training data into a machine learning algorithm to model clinical outcomes under different drug mixes and dose regimens. In various embodiments, the machine learning algorithm may be based on a regression model. In various embodiments, the regression model may be used for both prediction and understanding relationships between variables. In various embodiments, the regression model may be trained on historical data, learn patterns from the training data, and then make predictions on new, unseen data. In various embodiments, the regression model may be capable of predicting the clinical outcome of a drug based on a dose-response curve of the drug. In various embodiments, a dose-response curve of a drug illustrates the relationship between the dosage administered and the corresponding effect (e.g., physiological or pharmacological response) observed, e.g., measured. Typically, the dose-response curve is sigmoidal in shape, showing a gradual increase in response as the dosage rises, reaching a plateau where further increases in dosage yield diminishing returns. In various embodiments, training the AI model does not assume a specific mathematical form of the dose-response curve (e.g., sigmoidal). It would be appreciated that such a relationship cannot be assumed when non-parametric machine learning models are used.

In various embodiments, training the AI model may further comprise evaluating performance of the AI model (e.g., performance for regression, classification etc.) in relation to ground truth data using one or more metrics. Examples of metrics for evaluating the AI model include but are not limited to R², which is a statistical measure that represents the goodness of fit of a regression model, and AUC (Area Under the ROC Curve), which is a metric used in machine learning to evaluate the performance of a classification model. In various embodiments, evaluating performance of the AI model may comprise performing causality checks to determine whether predictions corresponding to counterfactual prescription regimens are sensible.

In various embodiments, training the AI model may further comprise periodically updating the retrospective data to iteratively refine the trained AI model. For example, the retrospective data may be updated on a quarterly basis, half-yearly basis, or yearly basis.

### Trained AI Model for Predicting Clinical Outcomes

In various embodiments, the method comprises parsing a selected subset of the list of treatment regimens through a trained artificial intelligence (AI) model to obtain predicted clinical outcomes for each of the selected subset of the list of treatment regimens. In various embodiments, the trained AI model may be a prediction model configured to forecast/predict the clinical outcome of a treatment regimen using the subject's visit profile and the treatment regimen as input. In various embodiments, the trained AI model may be configured to predict glycemic outcomes associated with a treatment regimen for diabetes.

In various embodiments, the subject's visit profile may include but is not limited to a previous visit treatment regimen, demographic data of the subject (e.g., age, gender, ethnicity), pre-visit measurements or recordings of laboratory tests, vitals, and diagnoses. For example, where the medical condition is diabetes, the subject's visit profile may comprise or consist of the previous visit treatment regimen information, patient demographic data, recent measurements of labs and vitals, information on diagnoses of comorbidities and complications of type 2 diabetes made during previous inpatient and outpatient visits. In various embodiments, the trained AI model may be configured to predict clinical outcome of a treatment regimen according to different time horizons. In various embodiments, the trained AI model may be configured to predict short term clinical outcome of a treatment regimen from about 1 month to about 12 months, from about 2 months to about 11 months, from about 3 months to about 10 months, from about 4 months to about 9 months, from about 5 months to about 8 months, or from about 6 months to about 7 months post-visit. In various embodiments, the trained AI model may be configured to predict long term clinical outcome of a treatment regimen from about 1 year to about 7 years, from about 2 years to about 6 years, or from about 4 years to about 5 years. For example, where the medical condition is diabetes, a typical range for blood glucose prediction may be from about 3 months to about 6 months; a typical range for hypoglycemia prediction may be about 6 months; and a typical range for occurrence of long-term complications may be from about 5 years to about 6 years.

In various embodiments, the input treatment regimen may be a treatment regimen from the selected subset of the list of treatment regimens generated by the optimizer. That is, the treatment regimen from the selected subset may be considered as a candidate for the one or more recommended treatment regimens to be generated by the presently disclosed method. In various embodiments, the previous visit treatment regimen may be represented in the form of the first vector. That is, the previous visit treatment regimen is represented as a vector having a length equal to the number of generic name drugs for the medical condition, wherein each entry in the vector represents a periodic dose of a corresponding generic name drug. In various embodiments, the input treatment regimen may be represented in the form of the second vector. That is, the recommended treatment regimen is represented as a vector having a length equal to the number of generic name drugs for the medical condition, where each entry in the vector represents a change in the periodic dose of a corresponding generic name drug at the second visit treatment regimen.

In various embodiments, obtaining predicted clinical outcomes for each of the selected subset of the list of treatment regimens may further comprise quantifying the uncertainty of the clinical outcomes by computing confidence intervals associated with the clinical outcomes. In various embodiments, the uncertainty of the clinical outcomes may be quantified, for example by using Bayesian neural networks, neural networks with Monte Carlo dropout as a Bayesian approximation or U-statistic-based random forests.

### Functions

In various embodiments, there is provided a plurality of functionalities that leverages the clinical knowledge database.

In various embodiments, there is provided a GUIDELINES function that is configured to generate a list of feasible medication changes, wherein the feasible medication changes are based on abstracted medical guidelines. In various embodiments, the GUIDELINES function is configured to receive the subject's medical profile and current treatment regimen as input. In various embodiments, each feasible medication change in the list comprises or consists of a current medication, a new medication and an action associated with the current and/or new medication. In various embodiments, the size of the list of feasible medication changes output by the GUIDELINES function depends on the number of generic name drugs for the medical condition and the number of medication changes recommended to be made. For example, if the medical guidelines recommend at most 1 medication change, the size of the output of the GUIDELINES function, i.e., the set of feasible medication changes, is of order O(N_{d}²) where N_{d} is the number of generic name drugs. Without this assumption, the set of potential medication changes (even ignoring dose) would be O(2^{N}_{d}), i.e., exponential in N_{d}.

In various embodiments, there is provided a DOSE LIST function that is configured to output an ordered list of feasible doses of a medication. In various embodiments, the DOSE LIST function is configured to receive the subject's medical profile and a generic name medication. In various embodiments, the ordered list of feasible doses of the medication is determined by availability at the formulary and renal dosing guidelines.

In various embodiments, there is provided a COST function that is configured to output a cost (in dollars) of an input treatment regimen.

In various embodiments, there is provided a PREF function that is configured to output a preference score based on one or more of (1) indicator functions of whether a prioritized medication is prescribed, and the negatives of indicator functions of whether a de-prioritized medication is prescribed; (2) linear functions in the dose of each medication; and (3) indicator functions of whether each medication is prescribed. In various embodiments, component (1) reflects medication prioritization and de-prioritization due to health benefits and risks. In various embodiments, component (2) reflects preferences for lower doses of drugs to be prescribed. In various embodiments, component (3) reflects preferences for fewer drugs to be prescribed if possible. In various embodiments, the higher the preference score of a treatment regimen, the more preferred is the treatment regimen. In various embodiments, the associated preference score for each of the selected subset of treatment regimens is based on indicator functions of whether a prioritized medication is prescribed; linear functions in the dose of each medication; and indicator functions of whether each medication is prescribed. For example, in the context of type 2 diabetes treatment, the PREF function may be configured as an indicator function for whether sodium-glucose cotransporter-2 inhibitor (SGLT2-i) is prescribed when the subject has been diagnosed with heart failure or chronic kidney disease, as there is strong evidence on the benefits of prioritizing SGLT2-i in patients with cardiovascular diseases.

In various embodiments, the COST function may be linear and increases with the dose of each generic name medication in the input treatment regimen, as higher doses are obtained by taking multiple tablets of a single given dose. In various embodiments, the PREF function may be linear and decreases with the dose of each generic name medication, between the minimum and maximum dose of each generic name medication. These characteristics of the COST and PREF functions may be applied to make the search for an optimal drug-mix and dose regimen more computationally tractable.

### Drug Mix and Dose Optimizer

In various embodiments, the optimizer is configured to parse pre-processed data of the subject to obtain a list of feasible treatment regimens. In various embodiments, the optimizer is configured to parse a selected subset of the list of feasible treatment regimens through a trained artificial intelligence (AI) model to obtain predicted clinical outcomes for each feasible treatment regimen of the selected subset of the list of feasible treatment regimens. In various embodiments, the optimizer is configured to obtain an associated cost for each feasible treatment regimen of the selected subset of the list of feasible treatment regimens. In various embodiments, the optimizer is configured to obtain an associated preference score for each feasible treatment regimen of the selected subset of the list of feasible treatment regimens. In various embodiments, the optimizer is configured to generate an associated value score for each feasible treatment regimen of the selected subset of the list of treatment regimens, said associated value score based on the predicted clinical outcomes, associated costs, and associated preference scores of the selected subset of the list of feasible treatment regimens. In various embodiments, the optimizer is configured to generate one or more recommended treatment regimens based on the associated value scores.

In various embodiments, parsing the pre-processed data of the subject through the optimizer to obtain the list of feasible treatment regimens may comprise accessing a clinical knowledge database to obtain the list of feasible treatment regimens from a broader list of potential treatment regimens. In various embodiments, the list of feasible treatment regimens may represent treatment regimens capable of achieving the clinical outcome (e.g., capable of achieving a specific HbA1c level), treatment regimens that involve a specific range of increment in treatment intensity (e.g., one or two levels) relative to the subject's current treatment regimen, and/or treatment regimens that involve a specific range of medication changes (e.g., one medication change via addition of a new medication, substitution of a current medication with a new medication, or increment in dosage of a current medication). In various embodiments therefore, treatment regimens that are not capable of achieving the clinical outcome, treatment regimens having treatment intensities that are higher than the specific range of increment in treatment intensity (e.g., an increase of more than two levels), and/or treatment regimens that involve medication changes higher than the specific range of medication changes (e.g., more than one medication changes), that are present in the broader list of potential treatment regimens may be filtered out from the list of feasible treatment regimens. In various embodiments, the list of feasible treatment regimens may be an ordered/ranked list of feasible treatment regimens.

In various embodiments, generating an associated value score for each treatment regimen in the selected subset of treatment regimens may comprise calculating a predicted percentage improvement in clinical outcome for the treatment regimen, calculating a weighted percentage reduction in associated cost, and calculating a weighted version of the associated preference score of the treatment regimen in the selected subset of treatment regimens. In various embodiments, generating the associated value score for each of the selected subset of treatment regimens may be based on an optimization of the following VALUE function/equation:

$$value ( Rec ) = \frac{{H}_{c} - PRED ( P , Rec )}{{H}_{c}} + {w}_{o} \frac{COST ( {Rec}_{curr} ) - COST ( Rec )}{COST ( {Rec}_{curr} )} + {w}_{1} PREF ( P , Rec )$$

wherein, P is a patient visit profile, Rec_{curr} is a current treatment regimen, Rec is a recommended treatment regimen, H_{c} is a pre-visit measurement of the clinical outcome, PRED is a clinical outcome prediction function of the trained AI model which outputs a predicted clinical outcome based on an input treatment regimen, PREF is a preference scoring function which outputs a score of an input treatment regimen, COST is a cost function which outputs a cost of an input treatment regimen, w₀ is a hyperparameter which controls a trade-off between improvement in clinical outcome and cost, w₁ is a hyperparameter which controls a trade-off between improvement in clinical outcome and a preferred medication prescription.

In various embodiments, generating one or more recommended treatment regimens may be based on obtaining one or more treatment regimens that optimizes the value function.

In various embodiments, optimizing the value function may involve maximizing the value function, i.e., maximizing the associated value score derived from the value function. In various embodiments, optimizing the value function may involve achieving an associated value score falling within a specific range of values.

In various embodiments, the optimizer utilizes a search heuristic that harnesses both the trained AI model and the clinical guidelines in the clinical knowledge database in order to make a recommendation that balances the trade-off among maximizing improvement in predicted clinical outcome, lowering costs, and recommending preferred medications. These three objectives are represented in the above value function as shown below.

$$Maximizing improvement in clinical outcome \text{:} \frac{{H}_{c} - PRED ( P , Rec )}{{H}_{c}}$$

$$Lowering cost \text{:} {w}_{o} \frac{COST ( {Rec}_{curr} ) - COST ( Rec )}{COST ( {Rec}_{curr} )}$$

Preferred Medication Prescription: w₁PREF(P, Rec)

In various embodiments, the search heuristic of the optimizer takes as input 2 hyperparameters: w₀, which controls the HbA_{1c} reduction-cost tradeoff, and w₁, which controls the HbA_{1c} reduction-preferred medication prescription tradeoff, where w₀ and w₁ can both take on any non-negative real values. By expressing health benefits and costs as percentages relative to the values taken in the previous visit treatment regimen, this problem formulation advantageously allows non-comparable quantities to be compared. In various embodiments, the hyperparameters w₀ and w₁ can be tuned to place more (or less) weight on the different objectives at play. In various embodiments, generating one or more recommended treatment regimens is based on a predicted percentage improvement in clinical outcome, and a weighted percentage reduction in associated cost. In various embodiments, generating one or more recommended treatment regimens is based further on a weighted version of the associated preference score of the recommended treatment regimens (obtained from a clinical knowledge database). In various embodiments, the weights w₀ and w₁ may be selected by a user.

In various embodiments, parsing the pre-processed data of the subject through the optimizer to obtain the list of feasible treatment regimens may comprise calling the function GUIDELINES to generate a set of feasible tuples {(m₁, m₂, a)}, where a∈{ADD, SUBSTITUTE, INCREASE DOSE} represents the action, m₁ represents the current treatment regimen medication (if any) to which the action is applied, and m₂ represents the new medication (if any) to which the action is applied.

In various embodiments, parsing the selected subset of the list of feasible treatment regimens through a trained AI model may comprise selecting, from the list of feasible treatment regimens, a list of feasible treatment regimens comprising a medication to be tuned. In various embodiments, parsing the selected subset of the list of feasible treatment regimens through a trained AI model may comprise sorting the list of feasible treatment regimens comprising the medication to be tuned, to obtain an ordered list of feasible doses of the medication to be tuned. In various embodiments, parsing the selected subset of the list of feasible treatment regimens through a trained AI model may comprise obtaining an order list of treatment regimens corresponding with the ordered list of feasible doses of the medication to be tuned. In various embodiments, parsing the selected subset of the list of feasible treatment regimens through the trained AI model may comprise, calling a DOSETUNE function to tune the dose of medication m₁ if the action a is INCREASE DOSE, and to tune the dose of medication m₂ if a is ADD or SUBSTITUTE. In various embodiments, the DOSETUNE function may further call a function DOSE LIST that can return an ordered list of feasible doses for each medication m, d₀, d₁, to d_{N(m, P)}, where d₀=0, d₁ is the minimum dose of medication m, and d_{N(m, P)} is the maximum dose of medication m for patient with profile P.

In various embodiments, parsing the selected subset of the list of feasible treatment regimens through a trained AI model may comprise, parsing the ordered list of treatment regimens to obtain an inflection treatment regimen associated with an inflection point of the value function, using a first binary search algorithm, and parsing the ordered list of treatment regimens between the inflection treatment regimen and a maximum treatment regimen to obtain one or more one or more recommended treatment regimens each having an associated value score that optimizes the value function. In various embodiments, parsing the ordered list of treatment regimens between the inflection treatment regimen and a maximum treatment regimen may be performed using a second binary search algorithm. In various embodiments, the first binary search algorithm may be performed to obtain an inflection point of the value function, in the ordered list of feasible doses. In various embodiments, the second binary search algorithm may be performed on the range between the inflection point and the maximum dose, to obtain the optimal dosage value and associated treatment regimen which maximizes the value function.

In various embodiments, instead of generating an associated value score for each of the treatment regimens in the list of feasible treatment regimens, associated value scores for a selected subset of treatment regimens from the list of feasible treatment regimens are generated. In various embodiments, the selected subset of treatment regimens may be selected based on a first binary search algorithm and a subsequent second binary search algorithm. In various embodiments, a binary search algorithm on a selected subset of treatment regimens may advantageously provide significant savings in computational cost as compared to a search heuristic that uses a brute force search to search among all possible combinations of medication changes.

In various embodiments, the treatment regimens in the list of feasible treatment regimens may involve at most one change. Accordingly, the treatment regimens in the selected subset of the list of feasible treatment regimens also involve at most one change. The one change may be a change in dosage of one drug, an addition of a drug from the treatment regimen, a removal of a drug from the treatment regimen or a replacement of a drug from the treatment regimen.

In various embodiments, for each feasible drug, the clinical outcome (e.g., HbA_{1c}) is assumed to be monotonically non-decreasing and convex in the dose up to a given dose, after which it will be monotonically non-decreasing and concave, i.e., there is an inflection point in the dose-clinical outcome curve.

In various embodiments, the COST function and PREF function are both linear in the dose of each generic drug, and in the range between minimum and maximum doses.

In various embodiments, for each generic drug, the optimal dose may be either 0 (i.e., the minimum dose), or at or after the inflection point.

In various embodiments, a binary search can be applied to the second differences in the value function VALUE to find the inflection point, based on the changing of the sign of the second differences. In various embodiments, the second differences in the VALUE function are the same as the second differences of the clinical outcome (e.g., HbA1c) reduction. In various embodiments, the second differences in the value function refer to the differences between the differences of the clinical outcome evaluated at consecutive values of the dose (i.e., consecutive treatment regimens within an ordered list of dosages of a particular drug/medication).

In various embodiments, a binary search can be applied to the interval between the inflection point and the maximum dose to find the dose d′ within the interval that maximizes the VALUE function. Thereafter, VALUE is evaluated at 0 (i.e., the minimum dose), and d′ to obtain the dose that maximizes VALUE.

In various embodiments, the selected subset of the list of treatment regimens are selected to perform the first binary search for obtaining the inflection point and/or the second binary search applied to the interval between the inflection point and the maximum dose to find a dose d′ within the interval which maximizes the VALUE function.

In various embodiments, parsing a selected subset of the list of treatment regimens through a trained artificial intelligence (AI) model may comprise, parsing the ordered list of treatment regimens to obtain an inflection treatment regimen associated with an inflection point of the value function, using a first binary search algorithm, and parsing the ordered list of treatment regimens between the inflection treatment regimen and a maximum treatment regimen, using the second binary search algorithm, to obtain one or more recommended treatment regimens each having an associated value score that optimizes the value function.

In various embodiments, parsing a selected subset of the list of feasible treatment regimens through a trained AI model may comprise, selecting, from the list of feasible treatment regimens, a first list of feasible treatment regimens comprising a first medication; sorting the first list of feasible treatment regimens based on a dosage quantity of the first medication to obtain a first ordered list of feasible doses of the first medication; obtaining a first ordered list of treatment regimens corresponding with the first ordered list of feasible doses of the first medication; parsing the first ordered list of treatment regimens to obtain an inflection treatment regimen associated with an inflection point of the value function, using a first binary search algorithm; and parsing the first ordered list of treatment regimens between the inflection treatment regimen and a maximum treatment regimen, using a second binary search algorithm, to obtain one or more recommended treatment regimens each having an associated value score that optimizes the value function. In various embodiments, the first medication may be an existing medication that the subject is taking for the medical condition. In various embodiments, in the first list of feasible treatment regimens comprising the first medication, each feasible treatment regimen includes the first medication but is not limited to the first medication. That is, each feasible treatment regimen may include other medication(s). In various embodiments, the steps as described above seek to increase dose of an existing medication.

In various embodiments, parsing a selected subset of the list of feasible treatment regimens through a trained AI model may comprise, selecting, from the list of feasible treatment regimens, a second list of feasible treatment regimens that excludes the first medication; sorting the second list of feasible treatment regimens based on a dosage quantity of a second medication to obtain a second ordered list of feasible doses of the second medication; obtaining a second ordered list of treatment regimens corresponding with the second ordered list of feasible doses of the second medication; parsing the second ordered list of treatment regimens to obtain an inflection treatment regimen associated with an inflection point of the value function, using the first binary search algorithm; and parsing the second ordered list of treatment regimens between the inflection treatment regimen and a maximum treatment regimen of the second medication, using the second binary search algorithm, to obtain one or more recommended treatment regimens each having an associated value score that optimizes the value function. In various embodiments, the second medication may be a new medication that is being recommended to replace/substitute the first medication. In various embodiments, the second medication is not part of the subject's existing treatment regimen. Accordingly, the steps as described above seek to tune the dosage of the second medication in treatment regimens where the first medication is not present.

In various embodiments, parsing a selected subset of the list of feasible treatment regimens through a trained AI model may comprise, selecting, from the list of feasible treatment regimens, a third list of feasible treatment regimens comprising a specific dosage of the first medication; sorting the third list of feasible treatment regimens based on a dosage quantity of a third medication to obtain a third ordered list of feasible doses of the third medication; obtaining a third ordered list of treatment regimens corresponding with the third ordered list of feasible doses of the third medication; parsing the third ordered list of treatment regimens to obtain an inflection treatment regimen associated with an inflection point of the value function, using the first binary search algorithm; and parsing the third ordered list of treatment regimens between the inflection treatment regimen and a maximum treatment regimen, using the second binary search algorithm, to obtain one or more recommended treatment regimens each having an associated value score that optimizes the value function. In various embodiments, the third medication may be a new medication that is being recommended to be added to the first medication (i.e., to be administered in combination with the first medication). In various embodiments, the third medication is not part of the subject's existing treatment regimen. In various embodiments, the third list of feasible treatment regimens comprises the first medication at a specific dosage and the third medication. Accordingly, the steps as described above seek to tune the dosage of the third medication in treatment regimens where the first medication is present and the dosage of the first regimen is kept at a specific dosage, e.g., current dosage being taken by the subject.

In various embodiments, generating one or more recommended treatment regimens may comprise generating treatment regimens with increasing intensity in a stepwise manner, with the subject's existing treatment regimen as a starting point.

In various embodiments, generating one or more recommended treatment regimens may comprise tuning the dosage of an existing medication to obtain an optimal (e.g., minimal or lowest) dosage of the existing medication capable of predictably achieving the desired clinical outcome. If such a minimal dosage cannot be found (i.e., the existing medication cannot predictably achieve the desired clinical outcome, regardless of the dosage of the existing medication), the existing medication may be substituted with a new medication, where the dosage of the new medication is tuned to obtain an optimal (e.g., minimal or lowest) dosage capable of achieving the desired clinical outcome. If such a minimal dosage of the new medication still cannot be found (i.e., replacement of the existing medication with the new medication cannot predictably achieve the desired clinical outcome, regardless of the dosage of the new medication), the new medication may be added to the treatment regimen of the existing medication such that the dosage of the existing medication is maintained, where the dosage of the new medication is tuned to obtain an optimal (e.g., minimal or lowest) dosage capable of achieving the desired clinical outcome. If such a minimal dosage of the new medication still cannot be found (i.e., addition of the existing medication to the treatment regimen cannot predictably achieve the desired clinical outcome, regardless of the dosage of the new medication), the highest feasible dosage of the new medication, along with the existing dosage of the existing medication is provided as the recommended treatment regimen.

In various embodiments, generating one or more recommended treatment regimens may comprise generating one or more treatment regimens with lower dosages of an existing medication, followed by generating one or more treatment regimens with higher dosages of the existing medication, followed by generating one or more treatment regimens substituting the existing medication with a new medication, and followed by generating one or more treatment regimens adding a new medication to the existing medication.

In various embodiments, generating one or more recommended treatment regimens may comprise generating one or more treatment regimens with lower dosages of an existing medication. In various embodiments, the one or more treatment regimens with lower dosages of the existing medication are presented as the one or more recommended treatment regimens if said one or more treatment regimens are predicted to be capable of achieving the clinical outcome.

In various embodiments, generating one or more recommended treatment regimens may comprise generating one or more treatment regimens with higher dosages of an existing medication. In various embodiments, the one or more treatment regimens with higher dosages of the existing medication are presented as the one or more recommended treatment regimens if said one or more treatment regimens are predicted to be capable of achieving the clinical outcome.

In various embodiments, generating one or more recommended treatment regimens may comprise generating one or more treatment regimens substituting the existing medication with a new medication. In various embodiments, the one or more treatment regimens substituting the existing medication with the new medication are presented as the one or more recommended treatment regimens if said one or more treatment regimens are predicted to be capable of achieving the clinical outcome.

In various embodiments, generating one or more recommended treatment regimens may comprise generating one or more treatment regimens adding a new medication to the existing medication. In various embodiments, the one or more treatment regimens adding the new medication to the existing medication are presented as the one or more recommended treatment regimens if said one or more treatment regimens are predicted to be capable of achieving the clinical outcome. In various embodiments, if the one or more treatment regimens adding the new medication to the existing medication are predicted to be incapable of achieving the clinical outcome, then said one or more treatment regimens closest to achieving the clinical outcome are presented as the one or more recommended treatment regimens.

### Interactive Dashboard

In various embodiments, the method may further comprise displaying a first and second recommended treatment regimens, predicted clinical outcomes and associated costs via an interactive dashboard. In various embodiments, the first and second recommended treatment regimens are displayed to one or more users, e.g., clinical practitioner and patient. In various embodiments, the first and second recommended treatment regimens may be automatically generated from the one or more recommended treatment regimens based on their associated value scores that optimizes the value function.

In various embodiments, the first recommended treatment regimen may be based on an associated value score that optimizes the value function weighted towards a minimum-intensity treatment regimen that is capable of achieving or maintaining the clinical outcome at a desired level. In various embodiments, the first recommended treatment regimen may be generated by calling the optimizer with the first hyperparameter w₀ set to zero. That is, the first recommended treatment regimen may be generated based on an optimization of the value function with the hyperparameter w₀ set to zero. In various embodiments, the second recommended treatment regimen may be based on an associated value score that optimizes the value function weighted towards achieving a lower cost of treatment, e.g., a lower cost of treatment than the first recommended treatment regimen. In various embodiments, the second recommended treatment regimen may be generated by calling the optimizer with the first hyperparameter w₀ set to a positive value such that a positive cost weight is applied. That is, the second recommended treatment regimen may be generated based on an optimization of the value function with the hyperparameter w₀ set to a positive value.

In various embodiments, the method may further comprise triggering a first selection on the interactive dashboard by a user to generate and display lower-cost/cheaper options/alternatives of treatment regimens. In various embodiments, the second recommended treatment regimen and optionally, the first recommended treatment regimen may be replaced with cheaper alternatives of treatment regimens. In various embodiments, both the first and second recommended treatment regimens may be replaced with cheaper alternatives of treatment regimens. In various embodiments, triggering the first selection on the interactive dashboard by a user may generate a third recommended treatment regimen representing a cheaper treatment regimen, to facilitate easy visual comparison with the first and second recommended treatment regimens. In various embodiments, the first selection may be configured to be repeatedly triggered to iteratively generate increasingly cheaper option(s) of treatment regimen(s) until an acceptable alternative or the cheapest available alternative is found. In various embodiments, for each successive iteration by triggering the first selection, an increasing cost weight is applied by increasing the first hyperparameter w₀.

In various embodiments, the generation of the first and second recommended treatment regimens and lower-cost alternatives thereof may be facilitated by the search heuristic of the optimizer. In various embodiments, the optimizer may be configured to harness both the trained AI prediction model and the clinical knowledge base to make a recommendation that balances the trade-off among maximizing the clinical outcome, lowering costs, and recommending preferred medications given weights that control the trade-off among these objectives. In various embodiments, the optimizer may be configured to perform multi-objective optimization in a computationally efficient manner, using binary search algorithms.

For example, the method may comprise regenerating the second recommended treatment regimen displayed on the interactive dashboard in response to user input to generate a lower-cost alternative, said regenerating step comprising, increasing the value of the hyperparameter w₀; generating an associated value score for each of the selected subset of treatment regimens by optimizing the value function based on the increased value of the hyperparameter w₀; generating a lower-cost treatment regimen based on the associated value score; and presenting the lower-cost treatment regimen as the regenerated second recommended treatment regimen; wherein the regeneration step is configured to be triggered iteratively by the user until the lowest-cost option is presented as the regenerated second recommended treatment regimen.

In various embodiments, the method may further comprise triggering a second selection on the interactive dashboard to simulate clinical outcomes of one or more alternative/customized treatment regimens based on information manually selected or entered by a user. In various embodiments, simulating clinical outcomes of one or more customized treatment regimens may comprise selecting one or more generic name drugs from a menu/list, e.g., drop-down menu and setting dosage and frequency of the one or more generic name drugs, e.g., by using free-text entry, and/or numerical data entry. In various embodiments, simulating clinical outcomes of one or more customized treatment regimens may further comprise extracting a numerical drug frequency from the frequency entered by the user. In various embodiments, simulating clinical outcomes of one or more customized treatment regimens may further comprise calculating periodic dosage(s), e.g., daily dosage(s), of the selected generic name drug(s) based on the dosage entered by the user. In various embodiments, simulating clinical outcomes of one or more customized treatment regimens may further comprise feeding the generic name drug(s) and dosage(s) into the trained AI model to output predicted clinical outcomes.

For example, the method may comprise simulating clinical outcomes of one or more alternative/customized treatment regimens based on manually entered user input, said simulation step comprising, preprocessing the one or more customized treatment regimens; parsing the one or more customized treatment regimens through the trained AI model to obtain predicted clinical outcomes for the one or more customized treatment regimens; and presenting the predicted clinical outcomes for the one or more customized treatment regimens via the interactive dashboard.

In various embodiments therefore, the second selection for simulating clinical outcomes of customized treatment regimens may advantageously facilitate exploration and comparison of customized treatment regimens.

### System for Generating Treatment Regimens for a Medical Condition

In various embodiments, there is provided a system for generating treatment regimens for a medical condition for a subject in need thereof, the system comprising, an optimizer for parsing pre-processed data of the subject to obtain a list of feasible treatment regimens; and a trained AI model for parsing a selected subset of the list of feasible treatment regimens to obtain predicted clinical outcomes for each of the selected subset of the list of feasible treatment regimens; wherein the optimizer is further configured to: obtain an associated cost for each of the selected subset of treatment regimens; obtain an associated preference score for each of the selected subset of treatment regimens; generate an associated value score for each of the selected subset of treatment regimens, said associated value score based on the predicted clinical outcomes, associated costs, and associated preference scores of the selected subset of the list of feasible treatment regimens; and generate one or more recommended treatment regimens based on the associated value scores, each recommended treatment regimen comprising a drug mix and dose regimen.

In various embodiments, the system is configured to receive EMR data of the subject as input for generating recommended treatment regimens. In various embodiments, the system is configured to receive configuration files that encode clinical knowledge for the optimizer to search for one or more recommended treatment regimens from the selected subset of the list of treatment regimens. In various embodiments, the system is configured to receive retrospective registry data for training the AI model.

In various embodiments, the system is configured to execute the presently disclosed method of generating treatment regimens for a medical condition for a subject in need thereof. In various embodiments, the system may comprise a plurality of modules that can be completely or partially realized as software modules running on a processor of a computer system.

### Module 1—Data Pre-Processor and Input Validation Processor

In various embodiments, the system comprises a first module comprising a data pre-processor and input validation processor. In various embodiments, the first module is configured to pre-process and validate input EMR data. In various embodiments, the data pre-processor and input validation processor of the presently disclosed system are configured to perform the various pre-processing and input validation functions described with respect to the presently disclosed method.

In various embodiments, the data pre-processor is configured to pre-process data of the subject by extracting one or more drug mixes and dose regimens or dose representations from an electronic medical record (EMR) of the subject.

In various embodiments, the data pre-processor is further configured to construct one or more configuration files based on historical EMR data of the subject. In various embodiments, the one or more configuration files may facilitate subsequent computation of vectors representing drug mixes and dose regimens.

In various embodiments, the data pre-processor is further configured to compute a first vector representing a drug mix and dose regimen of a first visit treatment regimen, e.g., previous visit treatment regimen. In various embodiments, the first vector has a length equal to the number of generic name drugs for the medical condition, and each entry in the first vector represents a periodic dose of a corresponding generic name drug. In various embodiments, the data pre-processor is further configured to compute a second vector representing a drug mix and dose regimen of a second visit treatment regimen, e.g., current visit treatment regimen. In various embodiments, the second vector has the same length as the first vector, and each entry in the second vector represents the periodic dose of a corresponding generic name drug in the second visit treatment regimen.

In various embodiments, the input validation processor is configured to perform a plurality of validation checks on the data of the subject to be pre-processed. In various embodiments, the validation checks advantageously ensure that the essential data fields are valid for downstream inference and recommendation generation.

In various embodiments, the input validation processor is further configured to trigger an alert message when a failure in one of the validation checks is detected. This allows a user, e.g., clinicians to be notified of the specific issue(s) so that clinicians can check on the input data at their end or proceed to make their own treatment recommendations to the patient. In various embodiments therefore, the input validation advantageously ensures validity of inputs and enhances system safety and robustness.

### Module 2—Model Training, Inference and Optimizer

In various embodiments, the system further comprises a second module comprising a model training, inference, and optimizer. In various embodiments, the second module is configured to train an AI prediction model using the retrospective registry data, use the trained AI prediction model for inference and optimization, and output recommended treatment regimens. In various embodiments, the trained AI prediction model is communicatively coupled to the optimizer. In various embodiments, the trained AI model and optimizer of the presently disclosed system are configured to perform the various training, prediction, inference and optimization functions described with respect to the presently disclosed method.

In various embodiments, the AI model is configured to predict the clinical outcome using the subject's medical profile and a treatment regimen as input. In various embodiments, the AI model is trained by pre-processing training data comprising retrospective data from electronic medical records of a group of patients having the medical condition; and feeding the pre-processed training data into a machine learning algorithm to model clinical outcomes under different drug mixes and dose regimens. In various embodiments, the electronic medical records may comprise the patients' medical profile data, historical drug mixes and dose regimen data and associated clinical outcome for each historical drug mix and dose regimen.

In various embodiments, the optimizer is configured to generate an associated value score for each of the selected subset of treatment regimens based on the following VALUE function:

$$value ( Rec ) = \frac{{H}_{c} - PRED ( P , Rec )}{{H}_{c}} + {w}_{o} \frac{COST ( {Rec}_{curr} ) - COST ( Rec )}{COST ( {Rec}_{curr} )} + {w}_{1} PREF ( P , Rec )$$

wherein, P is a patient visit profile, Rec_{curr} is a current treatment regimen, Rec is a recommended treatment regimen, H_{c} is a pre-visit measurement of the clinical outcome, PRED is a clinical outcome prediction function of the trained AI model which outputs a predicted clinical outcome based on an input treatment regimen, PREF is a preference scoring function which outputs a score of an input treatment regimen, COST is a cost function which outputs a cost of an input treatment regimen, w₀ is a hyperparameter which controls a trade-off between improvement in clinical outcome and cost, w₁ is a hyperparameter which controls a trade-off between improvement in clinical outcome and a preferred medication prescription.

In various embodiments, the optimizer is configured to utilize a search heuristic that harnesses both the trained AI model and the clinical guidelines in the clinical knowledge database in order to make a recommendation that balances the trade-off among maximizing improvement in clinical outcome, lowering costs, and recommending preferred medications. In various embodiments, the optimizer is configured to generate an associated value score for each of the selected subset of treatment regimens based on a predicted percentage improvement in clinical outcome, a weighted percentage reduction in associated cost, and a weighted preference score of the selected subset of treatment regimens. In various embodiments, the optimizer is configured to obtain one or more treatment regimens that maximize the value function.

In various embodiments, the optimizer is configured to implement a search heuristic which takes as input 2 hyperparameters: w₀, which controls the HbA1c reduction-cost tradeoff, and w₁, which controls the HbA_{1c} reduction-preferred medication prescription tradeoff, where w₀ and w₁ can both take on any non-negative real values. By expressing health benefits and costs as percentages relative to the values taken in the previous visit treatment regimen, this problem formulation advantageously allows non-comparable quantities to be compared. In various embodiments, the hyperparameters w₀ and w₁ can be tuned to place more (or less) weight on the different objectives at play. In various embodiments, the optimizer is configured to generate one or more recommended treatment regimens based on a predicted percentage improvement in clinical outcome, and a weighted percentage reduction in associated cost. In various embodiments, the optimizer is configured to generate one or more recommended treatment regimens based further on a weighted preference score of the recommended treatment regimens (obtained from a clinical knowledge database).

In various embodiments, the weights w₀ and w₁ may be selected by a user.

In various embodiments, the optimizer is configured to access a clinical knowledge database to obtain a list of feasible medication changes, wherein the list of feasible medication changes is an ordered list of feasible doses of a medication.

In various embodiments, the optimizer is configured to obtain an ordered list of treatment regimens corresponding with the ordered list of feasible doses.

In various embodiments, the optimizer is configured to parse the ordered list of treatment regimens to obtain an inflection treatment regimen associated with an inflection point of the value function, using a first binary search algorithm, and to parse the ordered list of treatment regimens between the inflection treatment regimen and a maximum treatment regimen to obtain one or more optimal treatment regimens as the one or more recommended treatment regimens.

In various embodiments, the optimizer may be configured to parse the ordered list of treatment regimens between the inflection treatment regimen and a maximum treatment regimen to obtain one or more optimal treatment regimens as the one or more recommended treatment regimens using a second binary search algorithm.

In various embodiments, the one or more optimal treatment regimens maximizes the value function:

$$value ( Rec ) = \frac{{H}_{c} - PRED ( P , Rec )}{{H}_{c}} + {w}_{o} \frac{COST ( {Rec}_{curr} ) - COST ( Rec )}{COST ( {Rec}_{curr} )} + {w}_{1} PREF ( P , Rec )$$

In various embodiments, the optimizer is configured to parse the pre-processed data of the subject to obtain a list of treatment regimens by calling the function GUIDELINES to generate a set of feasible tuples {(m₁, m₂, a)}, where a∈{ADD, SUBSTITUTE, INCREASE DOSE} represents the action, m₁ represents the current treatment regimen medication (if any) to which the action is applied, and m₂ represents the new medication (if any) to which the action is applied. In various embodiments, the optimizer is configured to call a DOSETUNE function to tune the dose of medication m₁ if the action a is INCREASE DOSE, and to tune to dose of medication m₂ if a is ADD or SUBSTITUTE. This DOSETUNE function further requires a function DoseList that can return an ordered list of feasible doses for each medication m, d₀, d₁, to d_{N(m, P)}, where d₀=0, d₁ is the minimum dose of medication m, and d_{N(m, P)} is the maximum dose of medication m for patient with profile P.

In various embodiments, instead of generating an associated value score for each of the treatment regimens associated with the ordered list of feasible doses, the optimizer may be configured to generate associated value scores for a selected subset of treatment regimens. The subset of treatment regimens is selected based on a first binary search algorithm and a subsequent binary search algorithm.

In various embodiments, the first binary search algorithm is configured to obtain an inflection point of the value function, in the ordered list of feasible doses. In various embodiments, the second binary search algorithm is configured to obtain the optimal dosage value and associated treatment regimen which maximizes the value function, on the range between the inflection point and the maximum dose.

In various embodiments, the selected subset of the list of treatment regimens are selected to perform the first binary search for obtaining the inflection point and/or the second binary search applied to the interval between the inflection point and the maximum dose to find a dose d′ within the interval which maximizes the VALUE function.

In various embodiments, feasible treatment recommendations involve at most one change. The one change may be a change in dosage of one drug, or an addition or removal of a drug from the treatment regimen.

In various embodiments, for each feasible drug, the clinical outcome (e.g., HbA_{1c}) is monotonically non-decreasing and convex in the dose up to a given dose, after which it will be monotonically non-decreasing and concave, i.e., there is an inflection point in the dose-clinical outcome curve.

In various embodiments, the COST function and PREF function are both linear in the dose of each generic drug, and in the range between a minimum and maximum doses.

In various embodiments, for each generic drug, the optimal dose must be either 0 (i.e., the minimum dose), or at or after the inflection point.

In various embodiments, the optimizer may be configured to apply a binary search to the second differences in the value function VALUE to find the inflection point, based on the changing of the sign of the second differences. In various embodiments, the second differences in the VALUE function are the same as the second differences of the clinical outcome (e.g., HbA1c) reduction. In various embodiments, the “second differences” is defined as the difference between the differences of a quantity of interest as evaluated at consecutive values of the dose (i.e., consecutive treatment regimens within an ordered list of dosages of a particular drug/medication). In this case, the quantity of interest is either the value function VALUE or the HbA1c reduction.

In various embodiments, the optimizer may be configured to apply a binary search to the interval between the inflection point and the maximum dose to find the dose d′ within the interval that maximizes the VALUE function. Thereafter, VALUE is evaluated at 0 (i.e., the minimum dose), and d′ to obtain the dose that maximizes VALUE.

### Module 3—Post-Processor

In various embodiments, the system further comprises a third module comprising a post-processor. In various embodiments, the post-processor performs post-processing of the treatment regimen recommendations, and the output of the post-processor is fed into an interactive dashboard user interface.

### Module 4—Binary Executable

In various embodiments, the system further comprises a fourth module comprising a binary executable. A binary executable, often simply referred to as an “executable” or “binary,” is a compiled file containing machine code that a computer's operating system can directly execute. In various embodiments, the first, second and third modules may be comprised within the fourth module. In various embodiments, the fourth module is configured to perform integration, compilation and encryption of the first, second and third modules.

### Module 5—Evaluation Processor

In various embodiments, the system further comprises a fifth module comprising an evaluation processor. In various embodiments, the evaluation processor is configured to monitor the performance of the system and trained AI prediction model. In various embodiments, the evaluation processor is further configured to serve as a feedback loop for model versioning. It would be appreciated that the performance of the trained AI model can drop due to various reasons, including changes in distribution of target data, updated clinical guidelines, changes of hospital management decision strategy, etc. In various embodiments therefore, the trained AI model is continuously monitored and updated to ensure that the system can perform reliably and consistently.

### User Interface (UI)

In various embodiments, the system further comprises a user interface in the form of an interactive dashboard configured to display a first and second recommended treatment regimens, predicted clinical outcomes and associated costs. In various embodiments, the interactive dashboard of the presently disclosed system is configured to perform the various functions of the interactive dashboard described with respect to the presently disclosed method. In various embodiments, the first and second recommended treatment regimens are displayed to one or more users, e.g., clinical practitioner and patient. In various embodiments, the first and second recommended treatment regimens may be automatically generated from the one or more recommended treatment regimens based on the predicted clinical outcomes and associated costs of the selected subset of the list of treatment regimens. Each treatment recommendation of the system comprises one or more medications identified based on their generic names; dosage of each of the one or more medications; cost of the one or more medications; and a predicted level of the marker together with an uncertainty estimate if the one or more medication were to be administered for a set period of time.

In various embodiments, the interactive dashboard further comprises a first selection configured to be triggered by a user to generate and display cheaper options of treatment regimens. In various embodiments, the first selection may be configured to be repeatedly triggered to iteratively generate increasingly cheaper option(s) of treatment regimen(s) until an acceptable option or the cheapest available option is found. In various embodiments, the interactive dashboard further comprises a second selection configured to be triggered by the user to simulate clinical outcomes of one or more treatment regimens manually entered by the user.

In various embodiments, all 5 modules of the system may be deployed centrally, using a centralized architecture of a health cluster. In various embodiments, the dashboard may be accessible from each constituent healthcare site of the health cluster as a web application that is restricted by firewall or whitelisted IP addresses. In various embodiments, each module may be implemented and deployed independently (e.g., in separate servers or virtual machine (VM) instance) with its own persistent database, and the controller (business logic/processing) and the view/interface as data I/O representation. In various embodiments, each module may be isolated and only accessible via an application programming interface (API) which defines and controls the data input and output that are allowed based on the defined user roles used for the request.

In various embodiments, modules may be segregated according to different functionality, workflows (both user and data), running frequency, and end user access. Alternatively, if a shared database is required, the database can be isolated, and the controller of each module granted access only to required data. In various embodiments, the system may be integrated without substantially interrupting an existing clinical workflow or hospital system.

### BRIEF DESCRIPTION OF FIGURES

FIG. 1 is a schematic flowchart for illustrating a method of generating treatment regimens for a medical condition for a subject in need thereof in an example embodiment.

FIG. 2 is a schematic diagram of a system for generating treatment regimens for a medical condition for a subject in need thereof in an example embodiment.

FIG. 3 is a first schematic block diagram of a clinical decision support system in an example embodiment.

FIG. 4 is a second schematic block diagram of the clinical decision support system (CDSS) in the example embodiment. FIG. 4 provides an overview of the CDSS, including its inputs, outputs, five modules, and user interface (UI). The inputs include electronic medical record (EMR) data, retrospective registry (SDR) dataset, and configuration files that encode clinical knowledge.

FIG. 5 is a schematic diagram illustrating how an EMR prescription history of a patient can be processed into vectors representing doses and change in doses of each generic name drug in an example embodiment. For this patient, at the visit on 1 Jun. 2020, the previous visit treatment regimen corresponds to the prescription on 1 Jan. 2020, of 1.5 g B.D. (bis die, or twice a day) of metformin, i.e., 3000 mg/day metformin, and the current visit treatment regimen on 1 Jun. 2020 is 3000 mg/day metformin and 5 mg/day linagliptin, corresponding to a change of 5 mg/day linagliptin.

FIG. 6 is a plot of a sigmoidal dose-response curve in an example embodiment. Existing literature on medication dosing shows that dose-response curves typically exhibit this relationship, where medication effect is monotonically increasing in dose, and convex (has increasing differences) up to an inflection point, after which it is concave (has decreasing differences).

FIG. 7 is a schematic diagram illustrating a dashboard design with “Treatment Options” and “Simulate alternatives” tabs in an example embodiment. The “Treatment Options” tab presents a minimal change treatment recommendation required to improve glycemic outcomes, as well as a lower cost option. By clicking the “Find cheaper option” button, a user can interactively generate an option of even lower cost. In the “Simulate alternatives” tab, the user can enter an alternative prescription regimen and simulate the associated post-visit HbA1c.

FIG. 8 provides a quantitative comparison of post-visit glycemic outcomes under Diab-Rx and standard of care (SoC) in the test set. Comparisons focus on visits where Diab-Rx recommended a prescription change relative to the previous prescription.

FIG. 9A and FIG. 9B are plots of the mean HbA_{1c} benefit relative to the Standard of Care (SoC) as hyperparameters controlling the different tradeoffs are tuned in an example embodiment. In FIG. 9A, the cost weight w₀ is varied and the mean monthly cost difference relative to the SoC is shown. In FIG. 9B, the weight on the rate of prescription of preferred drugs w₁ is varied and the mean rate of prescription of preferred drugs relative to the SoC is shown.

FIG. 10 is a histogram showing a breakdown of the type of recommendation made by an optimizer across patients with pre-visit HbA1c greater than 7%, as the hyperparameter w₀ in Equation (2) is increased in an example embodiment. Recommendations are grouped according to the introduction (addition or substitution) of glucagon-like peptide-1 receptor agonist (GLP-1 RA), sodium-glucose cotransporter-2 inhibitor (SGLT2-i), sulphonylurea, other oral antidiabetic drugs (OAD), or injectables, as well as dose increases and no change/dropping existing medications. Within each bar corresponding to a specific cost weight, the groups are arranged in the following order from the bottom up: introduce GLP-1 RA, introduce SGLT2-i, introduce sulphonylurea, introduce other OAD, introduce other injectable, dose increase, and no change/drop. The SoC recommendation is given on the extreme right.

FIG. 11A to FIG. 11C are histograms showing breakdowns of Diab-Rx's recommendations stratified across test set patients by pre-visit HbA_{1c} (FIG. 11A: 7-8%, FIG. 11B: 8-10%, FIG. 11C: greater than 10%), as the hyperparameter w₀ in Equation (2) is increased in an example embodiment. Recommendations are grouped according to the introduction (addition or substitution) of GLP-1 RA, SGLT2-i, sulphonylurea, other oral antidiabetic drugs (OAD), or injectables, as well as dose increases and no change/dropping existing medications. Within each bar corresponding to a specific cost weight, the groups are arranged in the following order from the bottom up: introduce GLP-1 RA, introduce SGLT2-i, introduce sulphonylurea, introduce other OAD, introduce other injectable, dose increase, and no change/drop. The SoC recommendation is given on the extreme right.

FIG. 12 is a schematic drawing of a computer system suitable for implementing an example embodiment.

FIG. 13 is a schematic drawing of a distributed computing system environment for implementing an example embodiment.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural and electrical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

### Method and System for Generating Treatment Regimens for a Medical Condition

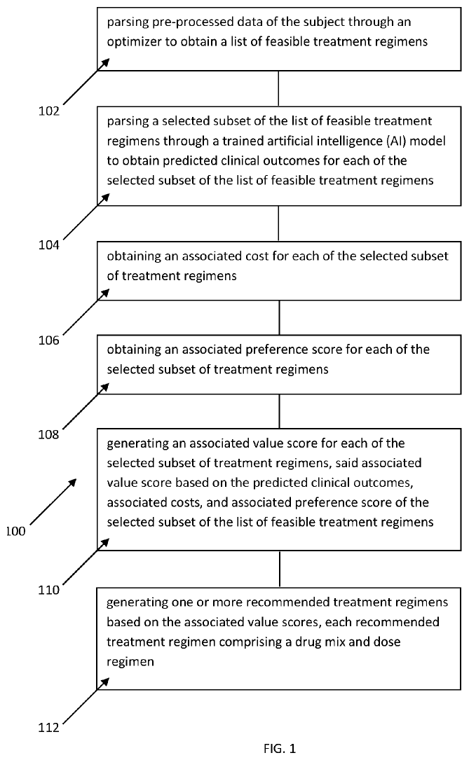

FIG. 1 is a schematic flowchart 100 for illustrating a method of generating treatment regimens for a medical condition for a subject in need thereof in an example embodiment. At step 102, pre-processed data of the subject is parsed through an optimizer to obtain a list of feasible treatment regimens. At step 104, a selected subset of the list of feasible treatment regimens is parsed through a trained artificial intelligence (AI) model to obtain predicted clinical outcomes for each of the selected subset of the list of feasible treatment regimens. At step 106, an associated cost for each of the selected subset of treatment regimens is obtained. At step 108, an associated preference score for each of the selected subset of treatment regimens is obtained. At step 110, an associated value score for each of the selected subset of treatment regimens is generated, said associated value score based on the predicted clinical outcomes, associated costs, and associated preference score of the selected subset of the list of feasible treatment regimens. At step 112, one or more recommended treatment regimens based on the associated value scores are generated, each recommended treatment regimen comprising a drug mix and dose regimen.

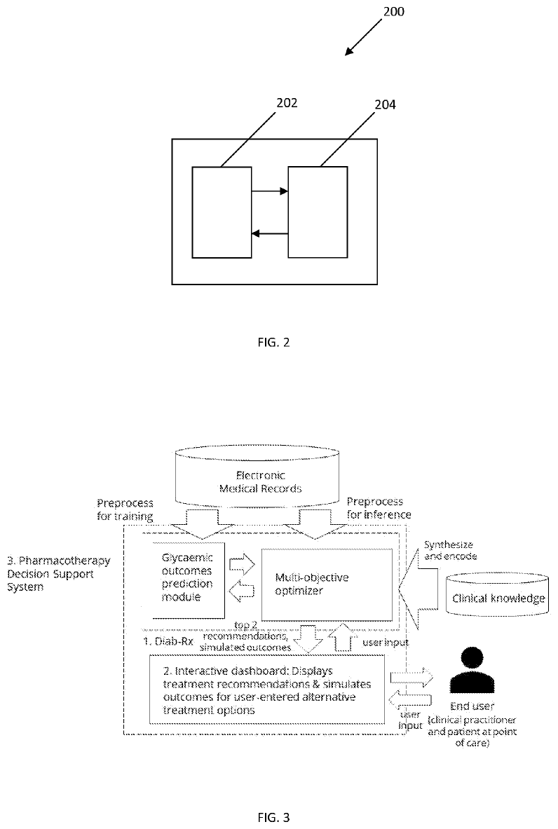

FIG. 2 is a schematic flowchart for illustrating a system 200 for generating treatment regimens for a medical condition for a subject in need thereof in an example embodiment. The system 200 comprises an optimizer 202 for parsing pre-processed data of the subject to obtain a list of feasible treatment regimens; and a trained AI model 204 for parsing a selected subset of the list of treatment regimens to obtain predicted clinical outcomes for each of the selected subset of the list of feasible treatment regimens. In the example embodiment, the optimizer 202 is further configured to: obtain an associated cost for each of the selected subset of treatment regimens; obtain an associated preference score for each of the selected subset of treatment regimens; generate an associated value score for each of the selected subset of treatment regimens, said associated value score based on the predicted clinical outcomes, associated costs, and associated preference scores of the selected subset of the list of feasible treatment regimens; and generate one or more recommended treatment regimens based on the associated value scores, each recommended treatment regimen comprising a drug mix and dose regimen.

Example embodiments of the presently disclosed method and system may provide an interactive treatment recommendation framework to address the unmet needs in clinical decision support applications. Example embodiments of the interactive treatment recommendation framework may have 3 main features. First, it embeds multi-objective optimization capability to generate and compare multiple granular recommendations that nearly optimize target outcomes but do so by favoring different trade-offs (e.g., cost, practicability). Second, it provides users the ability to interactively explore efficacy of alternative regimens that may suit clinician or patient preferences or tolerance levels. Third, it features a modular and robust decision support system design that is highly customizable for varying input data quality levels, end-user considerations, and/or integration requirements associated with distinct clinical use cases, settings, systems, and workflows. Collectively, these features serve to bring data-driven evidence and recommendations to the point-of-care in a manner that provides flexibility, considers practical factors such as those driving therapeutic inertia, and facilitates a shared decision-making process.

The presently disclosed method and system are instantiated for a Type 2 diabetes use case in the following example embodiments described below. In particular, the clinical outcome of interest is to achieve and maintain glycemic control with a view to reducing long-term complications. In the following example embodiments, an artificial intelligence drug mix and dose advisory, named Diab-Rx, uses data from an EMR to learn the relationship between treatment regimens and health outcomes, employs a heuristic to efficiently search a large space of viable treatment regimens for those that are most likely to address the outcomes of interest. The following example embodiments apply Diab-Rx on a real-world diabetes registry and showcase the advantages offered by the 3 features of the interactive decision support framework. The present disclosure has implications for real-world deployment and adoption of treatment recommendation systems for clinical decision support applications.

One difference between existing approaches and the presently disclosed approach lies in the choice of medication representation. While conventional approaches represented medications either at the subgroup or group level, the presently disclosed approach represents medications at the generic name level. This higher level of granularity allows the presently disclosed approach to extend the prescription recommendation problem beyond choice of drug mix (i.e., set of medications), and to also consider the dimension of dose. The optimization problem correspondingly becomes more complex, not only due to the much larger decision space, but also due to the changing objectives that come with considering dose titration: It is no longer sensible to select the medication regimen that minimizes blood glucose levels as this would correspond to simply setting doses at their maximum values. Instead, doses should be tuned by considering the minimal changes required to bring the patient's blood glucose levels into the target range, avoiding overly aggressive policies if possible.

### Clinical Decision Support System (CDSS)—Overall System Description

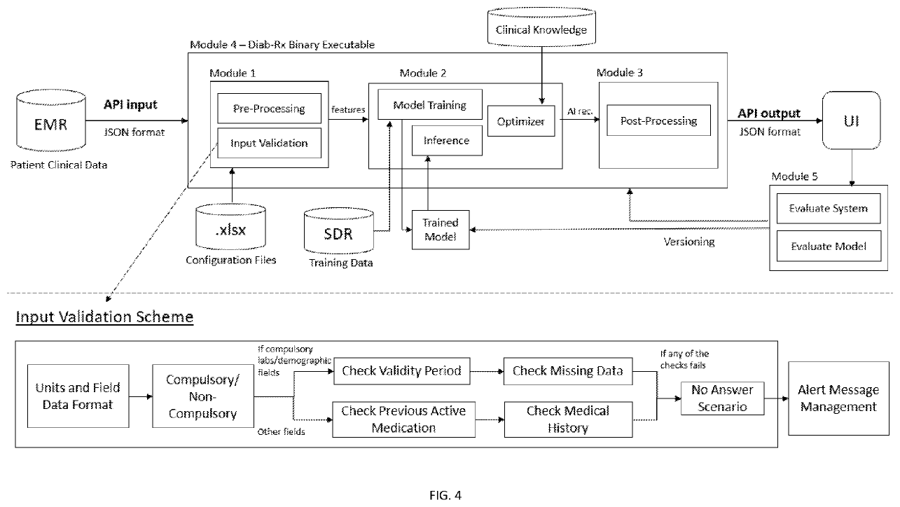

FIG. 3 outlines an interactive treatment recommendation framework comprising a clinical decision support system (CDSS) that takes as an input electronic medical records (EMR) data.

Training data is pre-processed to extract medical conditions and past prescription information at the drug mix and dose levels, then fed into a prediction model, e.g., glycemic outcomes prediction model, that is configured to learn to forecast blood glucose levels as a function of a patient's medical profile and recommended treatment regimen.

Inference data for incoming patients is similarly pre-processed to extract past prescription information and medical conditions, then fed into an optimizer that leverages the prediction model to compare and evaluate the outcomes of treatment alternatives in terms of both glycated hemoglobin (HbA_{1c}) reductions (given by the difference between the pre-visit HbA_{1c} and the HbA_{1c} outcome measured 2-6 months after the visit) and financial costs. Given hyperparameters that control the cost-HbA_{1c} reduction tradeoff, the optimizer is configured to apply a search heuristic to search a large space of potential drug mix and dose regimens and output a drug mix and dose regimen that optimizes this tradeoff. Together, these components (prediction model and optimizer) form an Artificial Intelligence drug mix and dose advisory (Diab-Rx).

At each patient visit, an interactive dashboard is configured to display two top recommendations to a user/end user, e.g., clinical practitioner: An HbA_{1c} lowering regimen that disregards costs, as well as a lower-cost option. An interactive feature is configured to allow the end user, e.g., patient, to express budget preferences by iteratively generating cheaper and cheaper options until an acceptable option (or the cheapest available option) is found. Another interactive feature is configured to allow the user to enter, simulate, and compare treatment alternatives from a wide range of potential drug-mix and dose regimens.

More details on implementation of the CDSS, prediction model, optimizer, and interactive dashboard tool are given below.

### CDSS Implementation

### User and Workflows

As shown in FIG. 3, end users of the system include the clinical practitioner and potentially the patient at point of care. Over time, each patient may visit multiple healthcare sites (defined as a hospital, specialty center, or primary care site where a patient has either received diabetes care or had a lab test for blood glucose). Patient records, including diagnoses, prescription history and laboratory test results are available in the centralized Electronic Medical Records (EMR) database, and are thus accessible at point of care. Other system users include system and deployment personnel (e.g., administrator or staff from a health system integrator), hospital management personnel, and data scientist/engineering personnel, each of whom may have different levels of access and interaction with the system.

The system has three main workflows and data streams: 1) model training 2) inference, and 3) user interactive adjustment. A registry comprising anonymized retrospective data from the EMR is used for model training. This is supplemented by clinical knowledge from other clinical databases. The retrospective data is updated regularly (e.g., on a yearly basis) to iteratively refine the model. Once the trained model is available, the inference workflow uses the optimizer, along with the trained model's blood glucose forecasts, to compare and evaluate viable options. This inference workflow may either be run periodically or in real-time based on user triggered action, generating recommendations that can be accessed and interactively adjusted by the end user. The user's final prescription selection can then be stored in the system. While the recommendations can be shared with the patient during a physical consultation, thus facilitating interactive adjustments, access is either limited to the clinical practitioner, or only granted to the patient at a read-only level (e.g., in a telemedicine setting.)

### Modules/Services

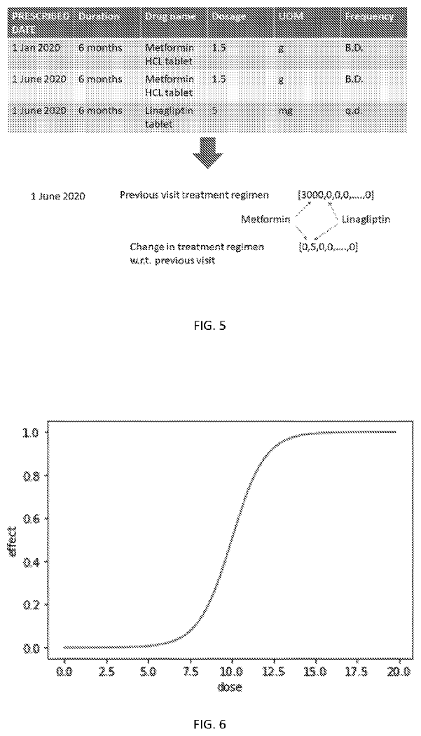

Based on the workflow, the system comprises the following main modules, pictured in FIG. 4: 1) data pre-processor and input validation processor, 2) model training, inference, and optimizer module, 3) post-processor, 4) a binary executable comprising the first three modules, and 5) an evaluation processor. The system takes as its input EMR data, configuration files that encode clinical knowledge, as well as retrospective registry data (denoted as SDR). The input EMR data is pre-processed and validated by Module 1. Module 2 trains an HbA_{1c} prediction model using the retrospective SDR data. It then uses this prediction model for inference and optimization, and output recommended treatment regimens. Module 3 performs post-processing of the recommendations, and the output is fed into an interactive dashboard user interface. Module 4 is the integration, compilation and encryption of Modules 1, 2 and 3. Finally, Module 5 performs performance monitoring of the system and prediction model, and serves as a feedback loop for model versioning. To ensure that the system can perform reliably and consistently, the model is continuously monitored and updated, as the model performance can drop due to various reasons, including changing distribution of the target data, updated clinical guidelines, changes of hospital management decision strategy, etc. More details on the data pre-processor, prediction model, and optimizer are given in the sections below.

All 5 main modules are deployed centrally, using a centralized architecture of a health cluster, and the dashboard is accessible from each of the constituent healthcare sites as a web application that is restricted by firewall or whitelisted IP addresses. Each module may be implemented and deployed independently (e.g., in separate server or virtual machine (VM) instance) with its own persistent database, and the controller (business logic/processing) and the view/interface as data I/O representation. Each module may also be isolated and only accessible via an application programming interface (API) which defines and controls the data input and output that are allowed based on the defined user roles used for the request. Modules are thus segregated according to different functionality, workflows (both user and data), running frequency, and end user access. Alternatively, if a shared database is required, the database can be isolated, and the controller of each module granted access only to required data. Integrating this system is unlikely to interrupt the existing clinical workflow or hospital system. Currently, the hospital system has interfaces that are used by the clinician and administrator to view patients' EMR. It may contain other modules, such as performance monitoring modules, and integration with telemonitoring devices, which offer functionalities beyond the scope of the presently disclosed system.

### Databases and Security

Minimal direct handling of patients' records decreases the risk of data privacy and security breaches. Hence, the system avoids duplicating patients' records within the different modules' data storage components wherever possible and also secures access to the required data by isolating the different modules. However, such isolation likely increases data storage needs, and correspondingly the burden of database maintenance and administration.

Of the various components of the CDSS listed above, only the interactive dashboard involves manual data entry from the end user. The training model primarily uses offline curated data from the EMR, while the inference processor requires online read-only access to the EMR database. The latter is accomplished by defining a user role with authentication attached to each EMR access request. Alternatively, each module's database access may be limited to certain IP addresses belonging only to the instances where the module is deployed. Since both the EMR data and the modules' other I/O data are well-defined structured data, a relational database (e.g., SQL) may be used to store the inference output and to capture end user's inputs.

### Data Pre-Processing

The data pre-processing routines contained within the data pre-processor and input validation processor (Module 1) and the model training, inference, and optimizer (Module 2) of the CDSS both condense complex and error-filled EMR data into representations of the patient profile and prescription history at each visit. Pre-processing routines are developed to automatically extract drug-mix and dose representations of the previous visit and current visit treatment regimens from prescription data.

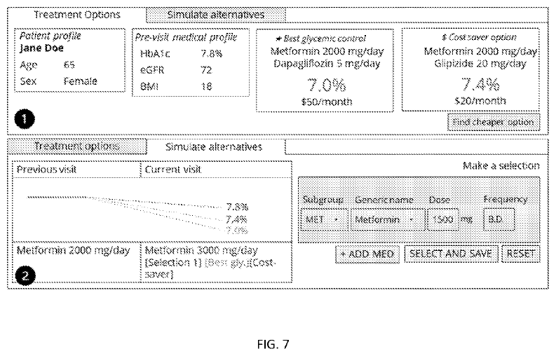

Previous visit treatment regimen is represented with vectors of length equal to the number of generic name drugs N_{d}, where each entry indicates the daily dose of the corresponding generic name drug. Current visit treatment regimen is represented with vectors of length N_{d}, where each entry indicates the change in daily dose of the corresponding generic name drug compared to the previous visit daily dose. An illustrative example of the pre-processing inputs and outputs is given in FIG. 5 below.

To compute these vectors, past EMR data is first used to construct configuration files that 1) map values on complex drug name information to generic name information, 2) map drug frequency information (typically listed in free text, using abbreviations of Latin terms) to numerical information, and 3) store typical doses (median, min, max) of different generic name medications.

Then, for each prescription visit, past prescriptions that are active at that visit date are determined by filtering out past prescriptions whose prescription windows (calculated as the duration from a prescribed date to the prescribed date plus a prescription duration and a grace period of 60 days) do not encompass the visit date.

Next, for each active past prescription, the configuration files are used to map drug name information to generic name drug information, and to map drug frequency information to numerical drug frequency information, and compute the daily dose for each generic name drug.

Finally, as drug information may be incorrectly entered (e.g., “milligrams” incorrectly entered instead of “grams”), a cleaning step is conducted to check if the dose obtained is of the same order of magnitude as the typical doses seen in the dataset. If a unit of measure (UOM) change results in a more appropriate magnitude, the UOM is changed accordingly.

### Input Validation Scheme

As Module 1 performs pre-processing of the raw EMR data to transform them into appropriate patient profile and prescription history representations, a plurality of validation checks is put in place to ensure that essential data fields are valid for downstream inference and recommendation generation. The validation checks are mainly focused on two aspects: checking for compulsory fields (e.g., demographics such as namely age, gender, and laboratory results such as creatinine serum, and HbA_{1c} lab results) and their validity period (for labs only); and checking for other fields such as checking for the previous prescription information. For the latter, the validation checks check for the following scenarios: 1) whether any past prescriptions were extracted, 2) whether the previous prescription has exceeded the validity period of 1 year, 3) whether the drug has been phased out or is a newly released drug, 4) whether the dosage information is valid (available and correct), and 5) whether the drug mix is contraindicated. If any one of the validation checks fails, Module 1 is configured to issue an alert message to notify clinicians the specific issue(s) so that the clinicians can check on the input data at their end, or proceed to make their own treatment recommendations to the patient. This scheme ensures validity of inputs and enhances system safety and robustness.

### Blood Glucose Prediction Model

The prediction model in the CDSS trains a regression model PRED with a blood glucose outcome—or a HbA_{1c} outcome—2-6 months after a visit as the dependent variable, and information on a recommended treatment regimen (Rec) and a patient visit profile (P) as the independent variables (Equation 1 below).

The recommended treatment regimen is represented by a vector of length equal to the number of generic name medications, and where each entry reflects a change in daily dose of a corresponding generic name medication relative to a previous visit recommended treatment regimen.

The patient visit profile comprises previous visit treatment regimen information, patient demographic data, recent measurements of labs and vitals, information on diagnoses of comorbidities and complications of type 2 diabetes made during previous inpatient and outpatient visits. See Equation (1) below.

$$HbA 1 c outcome \sim Recommended treatment regimen Rec , Patient visit profile P ( Previous visit treatment regimen , demographic data , previsit measurements or recordings of labs , vitals , diagnoses ) \qquad ( 1 )$$

Besides the HbA_{1c} outcome, the prediction model may also be configured to quantify uncertainty and compute associated confidence intervals, for example by using Bayesian neural networks, neural networks with Monte Carlo dropout as a Bayesian approximation or U-statistic-based random forests, to inform end user interpretation if needed.

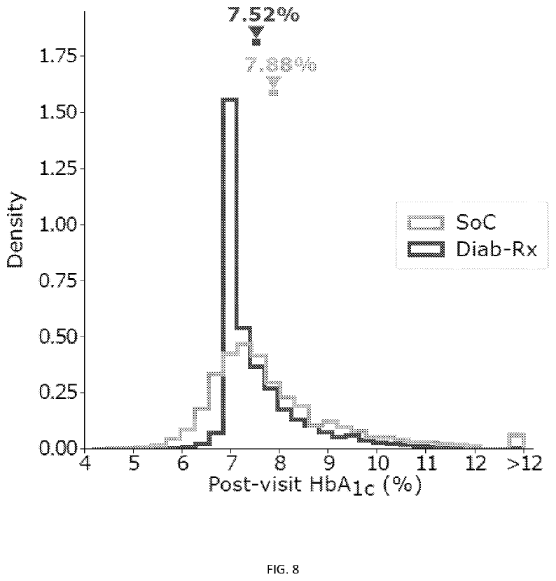

It is noted here that the relationship between the dose of each generic name medication and the HbA_{1c} outcome can be assumed to exhibit certain mathematical properties. For a wide variety of drug dosing scenarios, beyond just type 2 diabetes, it has been empirically observed that the relationship between dose and effect—as described by so-called dose-response curves—follows a sigmoidal curve, as shown in FIG. 6. That is, the dose-response curve is convex and increasing for low doses, and concave and decreasing for higher doses. In this setting, where the effect is given by the HbA_{1c} reduction, it is expected that the HbA_{1c} reduction is 1) monotonically increasing in the dose of each generic name medication, 2) convex and increasing up to some inflection point, 3) concave and increasing beyond this inflection point. Then, it can be assumed that a regression model PRED with high prediction accuracy would reflect these properties as well. The description of the Optimizer below shows how these three properties can be utilized to perform dose tuning, and to search for an optimal drug-mix and dose regimen in a more computationally tractable way. It should be noted that no specific mathematical form of the dose-response curve (e.g., sigmoidal) is assumed. Such a relationship cannot be assumed when non-parametric machine learning models are used to learn PRED.

### Clinical Knowledge Base

In practice, pharmacological treatment recommendations for type 2 diabetes patients are guided not only by blood glucose forecasts and costs, but also by clinical knowledge of the potential effects (both positive and negative) that different therapies may have on the patient's health condition. Medications with positive health benefits should be prioritized, while medications with negative health effects should be de-prioritized. Further, due to potential side effects, including hypoglycemia, a very severe condition resulting from low blood sugar levels, less intensive treatment regimens that can achieve a given blood glucose level are preferable to more intensive treatment regimens.

A relatively large body of clinical knowledge-based guidelines for type 2 diabetes pharmacotherapy from various sources is condensed into a compact but comprehensive set of rules to inform treatment regimen choices. Sources include the Health Sciences Authority (HSA) (https://www.hsa.gov.sg/), MIMS (https://www.mims.com/singapore/), the American Diabetes Association (ADA) (https://www.diabetes.org/), the European Association for the Study of Diabetes (EASD) (https://www.easd.org/), and knowledge of the available formularies.

The condensed set of guidelines generally fall into the following categories:
Medication contraindications: For safety reasons, certain medications should not be prescribed depending on the patient's health condition.
Certain combinations of medications should not be prescribed together, also for safety reasons.
Set of viable doses for each medication: The prescribed dose of each medication should belong to a discrete set, as determined by formulary availability (in the present case, at the local health services formulary).
Renal dosing: Depending on the patient's kidney function, the maximum viable dose of each medication may be limited.
The severity level increase resulting from a treatment recommendation should be capped at 1 to 2, to prevent too-sudden treatment intensification. The severity level corresponding to a treatment regimen takes on one of five potential values: 1—One oral medication only, 2—combination oral therapy, 3—GLP-1 RA or basal insulin, 4—premixed insulin, 5—basal bolus insulin.
At most 1 medication change, in the form of an addition, substitution, or dose increase, should be prescribed, in line with current clinical practice.
Medication prioritization/de-prioritization due to health benefits and risks: Some medications should be prioritized or de-prioritized based on the associated health benefits and risks. Some obsolete medications should be completely removed from the list of viable options.
Treatment regimens with lower treatment intensity should be prioritized. The treatment intensity is increasing in the severity level, in the doses of the constituent drugs, and the total number of prescribed drugs (i.e., medication additions are considered more intense than substitutions).

The knowledge base/bank also includes data on medication costs, which can be taken into consideration to include more budget-friendly options in the list of recommendations.

Based on these guidelines, the following functions are constructed. The functions are then used by the Optimizer to search for an optimal recommendation among potential recommendations:

GUIDELINES: P, Rec_{curr}→{(m₁, m₂, a)}

This function takes as inputs the patient medical profile P and current treatment regimen Rec_{curr}, gives as output a set of feasible medication changes {(m₁, m₂, a)}. For each medication change (m₁, m₂, a), a∈{ADD, SUBSTITUTE, INCREASE DOSE} represents the action, m₁ represents the current treatment regimen medication (if any) to which the action is applied, and m₂ represents the new medication (if any) to which the action is applied.

DOSE LIST: P, m→[dᵢ_{_{min′}}, . . . , dᵢ_{ₘₐₓ}]

This function takes as inputs the patient medical profile P and a generic name medication m, and gives as outputs a list of feasible doses in increasing order, where feasibility is determined both by availability at the formulary and renal dosing guidelines.

### COST: Rec→c

This function takes as input a treatment regimen Rec, and gives as output a cost (in dollars).

### PREF: P, Rec→s

This function takes as inputs the patient medical profile P and recommended treatment regimen Rec, and gives as output a score s. The greater the score, the more preferred the treatment regimen Rec. The function PREF is defined as the sum of (1) indicator functions of whether a prioritized medication is prescribed, and the negatives of indicator functions of whether a de-prioritized medication is prescribed; (2) linear functions in the dose of each medication; (3) indicator functions of whether each medication prescribed. Then components (1) reflect medication prioritization and de-prioritization due to health benefits and risks, components (2) reflect preferences for lower doses of drugs to be prescribed, while components (3) reflect the preference for fewer drugs to be prescribed if possible.

Some observations can be made about the mathematical properties of the above functions. First, since the clinical guidelines recommend at most 1 medication change, the size of the output of the GUIDELINES function, i.e., the set of feasible medication changes, is of order O(N_{d}²) where N_{d} is the number of generic name drugs. Without this assumption, the set of potential medication changes (even ignoring dose) would be O(2^{Nd}), i.e., exponential in N_{d}.

It is also observed empirically that the COST function is linear and increasing in the dose of each generic name medication in Rec, as higher doses are obtained by taking multiple tablets of a single given dose. By definition, the PREF function is also linear and decreasing in the dose of each generic name medication, between the minimum and maximum dose of each generic name medication. In the description of the Drug Mix and Dose Optimizer given below, it is shown how these properties can be applied to make the search for an optimal drug-mix and dose regimen more computationally tractable.

### Drug Mix and Dose Optimizer

A search heuristic is developed to harness both the blood glucose prediction model and the clinical guidelines in order to make a recommendation that balances the trade-off among maximizing the HbA_{1c} reduction, lowering costs, and recommending preferred medications. This heuristic takes as input 2 hyperparameters: w₀, which controls the HbA_{1c} reduction-cost tradeoff, and w₁, which controls the HbA_{1c} reduction-preferred medication prescription tradeoff, where w₀ and w₁ can both take on any non-negative real values. The heuristic then finds the treatment regimen Rec that optimizes Equation (2) below.

$${max}_{Rec : Rec is feasible} \frac{{H}_{c} - PRED ( P , Rec )}{{H}_{c}} + {w}_{o} \frac{COST ( {Rec}_{curr} ) - COST ( Rec )}{COST ( {Rec}_{curr} )} + {w}_{1} PREF ( P , Rec ) \qquad ( 2 )$$

Here, P is the patient visit profile, Rec_{curr} is the current treatment regimen, H_{c} is the pre-visit HbA_{1c} (measured within the past month of the visit), PRED is the HbA_{1c} prediction function as described in the “Blood glucose prediction model” section above, and PREF is the preference scoring function as defined in the “Clinical knowledge base” section above. By expressing health benefits and costs as percentages relative to the values taken in the previous visit treatment regimen, this problem formulation allows non-comparable quantities to be compared. The hyperparameters w₀ and w₁ can then be tuned to place more (or less) weight on the different objectives at play.

A full description of the search heuristic used to solve Equation (2) is given in Algorithm 1 below. The heuristic first calls the function GUIDELINES to generate the set of feasible tuples {(m₁, m₂, a)}, where a∈{ADD, SUBSTITUTE, INCREASE DOSE} represents the action, m₁ represents the current treatment regimen medication (if any) to which the action is applied, and m₂ represents the new medication (if any) to which the action is applied.

The heuristic then uses a novel DOSETUNE subroutine to tune the dose of medication m₁ if the action a is INCREASE DOSE, and to tune to dose of medication m₂ if a is ADD or SUBSTITUTE. This subroutine requires a function DoseList that can return an ordered list of feasible doses for each medication m, d₀, d₁, to d_{N(m, P)}, where d₀=0, d₁ is the minimum dose of medication m, and d_{N(m, P)} is the maximum dose of medication m for patient with profile P. Instead of brute-force searching this list (this would be linear in the number of doses), the subroutine consists of two consecutive binary search procedures, the first to find the inflection point of the value function, and the second to search for the optimal dose on the range between the inflection point and the maximum dose.

Since the computational complexity of a binary search is logarithmic in the size of the search space, the computational complexity of Algorithm 1 is then O(N_{d}² log(D)) where N_{d} is the number of generic name drugs and D is the maximum number of doses in any generic name medication's dose set. In comparison, a search heuristic that used a brute force search for dose tuning would have computational complexity O(N_{d}²D), while a search heuristic that searched among all possible combinations of medication changes would have computational complexity O(D^{N}^{_{d}}). In the present setting, N_{d} is 25 and D can be up to 70 (for some insulins, the maximum dose of a new medication is 70 units/day, and any integer-valued dose under this value can be considered). Thus, the savings in computational cost are significant.

The rationale behind this search heuristic is as follows: Firstly, as explained in the “Clinical knowledge base” section, it can be assumed that feasible treatment recommendations involve at most one change, as this mirrors clinical practice. Secondly, as discussed in the “Blood glucose prediction model” section, if PRED is learned accurately, for each generic name medication, the HbA_{1c} reduction will be monotonically non-decreasing and convex in the dose up to a given dose, after which it will be monotonically non-decreasing and concave, i.e., there is an inflection point in the dose-HbA_{1c} reduction curve.

As discussed in the “Clinical knowledge base” section, COST and PREF functions are both linear in the dose of each generic name medication, in the range between the minimum and maximum doses. Then, for each generic name medication, the optimal dose must be either 0 (i.e., the minimum dose), or a dosage value at or after the inflection point. To elaborate, suppose the optimal dose is instead greater than the minimum dose but less than the inflection point. Let this dose be dᵢ, i.e., it is the ith dose from the list of feasible doses. Then VALUE is greater at dᵢ than at dᵢ₋₁, i.e., the increase in HbA1c from increasing the dose from dᵢ₋₁ to dᵢ is greater than the sum of the increase in COST minus PREF. However, since this dose is less than the inflection point dose, i.e., HbA_{1c} reduction function is convex and has increasing differences, the increase in HbA_{1c} reduction from increasing the dose from dᵢ to dᵢ₊₁ is greater than the HbA_{1c} reduction from increasing the dose from dᵢ₋₁ to dᵢ. Since COST and PREF are linear at dᵢ₋₁, dᵢ, dᵢ₊₁, the change in COST minus PREF is exactly the same. Then VALUE would increase from dᵢ to dᵢ₊₁, giving rise to a contradiction.

A binary search can be applied to the second differences in the value function VALUE to find this inflection point, using the fact that the inflection point by definition is where the second differences change sign, as well as the fact that the second differences in VALUE are the same as the second differences of the HbA_{1c} reduction. The “second differences” is defined as the difference between the differences of a quantity of interest as evaluated at consecutive values of the dose (i.e., consecutive treatment regimens within an ordered list of dosages of a particular drug/medication). In this case, the quantity of interest is either the value function VALUE or the HbA1c reduction. Since the second differences of linear functions are 0, the second differences of VALUE are equal to the second differences of the HbA_{1c} reduction. Then, using the fact that the value function is concave after the inflection point, a binary search can be applied once again to the interval between the inflection point and the maximum dose to find the dose d′ on this interval that maximizes VALUE. Finally, VALUE is evaluated at 0 (i.e., the minimum dose), and d′ to obtain the dose that maximizes VALUE.

Algorithm 1: Make a treatment recommendation that optimizes the HbA_{1c} reduction-cost
tradeoff
Inputs: Patient visit profile P including current treatment regimen Rec_{curr} and pre-visit HbA_{1c}
H_{c}; function PRED with inputs profile P′ and treatment regimen Rec′, and output predicted
future HbA_{1c} outcome H_{f}; function GUIDELINES that takes profile P′ and current medications
Rec′_{curr} as inputs and outputs M′, a list of feasible potential new medications {(m₁, m₂, a)};
function PREF with inputs profile P′ and recommendation Rec′, and output score S;
hyperparameter w₀ controlling the HbA_{1c} reduction-cost tradeoff; hyperparameter w₁
controlling the HbA_{1c} reduction-preferred medication prescription tradeoff.
Outputs: Recommended treatment recommendation Rec.
VALUE(Rec) ← (PRED(P,Rec)−H_{c})/H_{c} + w₀.(COST(Rec)−COST(Rec_{curr}))/COST(Rec_{curr}) + w₁.
PREF(P,Rec)
M ← GUIDELINES(P, Rec_{curr})
Set Rec ← null, V_{f,best} ←infinity.
For each tuple (m₁, m₂, a) in M:
Rec′, V_{f} ← DOSETUNE((m₁, m₂, a), VALUE)
If V_{f} < V_{f,best}:
V_{f,best} ← V_{f}
Rec ← Rec′
End
End
Return Rec.
Subroutine DOSETUNE
Inputs: Medication change tuple (m₁, m₂, a), value function VALUE to maximize
Outputs: Recommended treatment recommendation Rec, treatment recommendation value
V
m ← m₁ if a == INCREASE DOSE and m ← m₂ if a ∈ {ADD, SUBSTITUTE}.
iₘᵢₙ ←1, iₘₐₓ ←|DoseList(m)|−1, i_{inflection} ←1
while iₘₐₓ − iₘᵢₙ > 2:
i_{mid}← FLOOR((iₘᵢₙ+ iₘₐₓ)/2)
Rec_{mid} ← REC(m₁, m₂, a, DoseList(m)[i_{mid}])
Rec_{mid+2} ← REC(m₁, m₂, a, DoseList(m)[i_{mid+2}])
V_{gap,mid} ← VALUE(Rec_{mid+2})−VALUE(Rec_{mid})
if V_{gap,mid} > 0:
iₘᵢₙ ← i_{mid}
else:
iₘₐₓ + i_{mid}
end
end
i_{inflection} ← iₘᵢₙᵢ
iₘᵢₙ ← i_{inflection}, iₘₐₓ ←|DoseList(m)|−1
while iₘₐₓ − iₘᵢₙ > 1
i_{mid}← FLOOR((iₘᵢₙ+ iₘₐₓ)/2)
Rec_{mid} ← REC(m₁, m₂, a, DoseList(m)[i_{mid}])
Rec_{mid+1} ← REC(m₁, m₂, a, DoseList(m)[i_{mid}+1])
V_{gap,mid} ← VALUE(Rec_{mid+1})−VALUE(Rec_{mid})
if V_{gap,mid} > 0:
iₘₐₓ = i_{mid}
else:
iₘᵢₙ = i_{mid}
end
end
Rec ← null, V ← infinity
For i in UNION(0, 1, RANGE(iₘᵢₙ, iₘₐₓ)):
Recᵢ < REC(m₁, m₂, a, DoseList(m)[i])
Vᵢ ← VALUE(Recᵢ)
If Vᵢ < V:
Rec ← Recᵢ, V ← Vᵢ
end
end
Return Rec, V

### Interactive Dashboard

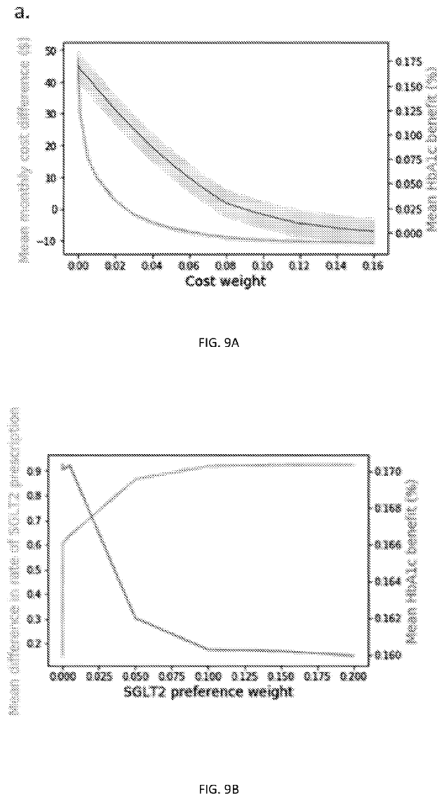

An interactive dashboard tool is designed to display Diab-Rx's recommendations to a user, e.g., clinical practitioner and patient (see FIG. 7).

The interactive dashboard tool comprises a main tab, labeled “Treatment Options,” which displays two automatically generated recommendations to the user, while also allowing the user to iteratively generate cheaper options if preferable. The “Treatment Options” tab displays the following two recommendations: 1) a minimal change treatment recommendation, generated by calling the optimizer with cost weight 0, and 2) a lower cost option, generated by calling the optimizer with some positive cost weight.

The “Treatment Options” tab also includes an interactive feature: a “Find cheaper option” button that can be clicked to call the optimizer with an increased cost weight and generate an option of even lower cost. If the generated option is still too expensive, the button can be clicked iteratively, calling the optimizer with increased cost weights each time, and generating options of lower cost until either an acceptable option or the cheapest available option has been found. It would be appreciated that the relatively low computational complexity of the search heuristic described in the “Drug mix and dose optimizer” section is important to this feature, as the optimizer may be called multiple times. This feature allows the patient to express complex budget preferences that include not only the amount he/she is willing to pay, but also the amount that he/she is willing to pay for a given health benefit. These preferences may be difficult to state explicitly, and the interactive feature allows the patient to express them without stating them explicitly, and to accordingly generate cheaper options.

The interactive dashboard tool further comprises a “Simulate alternatives” tab which provides an interactive tool that facilitates exploration and comparison of alternative treatment regimens/recommendations. Here, since these alternatives belong to a very large search space, it is not possible to enumerate all possible regimens and allow users to select from a given list. Instead, the design of the interactive dashboard allows users to enter alternative treatment regimens by first selecting generic name drugs from a drop-down menu, then by setting the drug dosage and frequency using free-text entry. This mode of entry gives the user full flexibility in entering a treatment regimen, while also ensuring that alternatives may be added and amended quickly and conveniently. Given the user input, a backend data pre-processor then extracts numerical drug frequency information from the entered prescription, calculates the daily dosages of the medications in the entered regimen, and applies the prediction model to output the associated post-visit HbA_{1c}.

As an illustrative example, consider the dummy patient depicted in FIG. 7. The top recommendation is to add 5 mg/day of dapagliflozin to a regimen consisting of 2000 mg/day metformin, which is expected to give HbA1c of 7.0%. If the clinician prefers to prescribe metformin at 1500 mg/day B.D. —where B.D. stands for bis die, meaning twice a day, i.e., this would constitute a dose increase of metformin from 2000 mg/day to 3000 mg/day—they can select this regimen and obtain a predicted HbA1c of 7.4%.

Comparison with Prior Art Approaches

A discussion of the main features of the presently disclosed CDSS, treatment advisory Diab-Rx, and interactive tool that (a) distinguish it from other related approaches for type 2 diabetes and (b) power it for safe and robust decision support, are provided below.

First, the treatment advisory, Diab-Rx, generates granular treatment regimen recommendations that not only take into account glycemic control targets (HbA_{1c}) but also multifactorial considerations such as cost and medication preferences. Diab-Rx has the capability to address the novel and challenging optimization problem of identifying the drug mix and dose regimen that would concomitantly optimize outcomes in alignment with a comprehensive set of clinical guidelines. Another novelty of the optimization problem formulation is that the objective expresses HbA_{1c} reductions and costs in percentages to allow non-comparable quantities to be compared, and uses weights as hyperparameters to balance the different tradeoffs. Diab-Rx then employs a novel search heuristic to solve this complex optimization problem in a computationally tractable way. Specifically, the search heuristic leverages clinical insights and other domain knowledge of the set of feasible recommendations, the mathematical properties of the dose-response curve, the linearity of the cost function, and factors that determine treatment intensity; and identifies minimal prescription changes required to achieve the targeted objectives.

Second, unlike other prior approaches, the presently disclosed approach embeds interactive optimization capabilities to dynamically adapt to user needs and preferences. Specifically, the presently disclosed approach allows the user (i.e., clinical provider) to (1) iteratively search for cheaper options quickly and conveniently, by calling Diab-Rx with increasing cost weights, and (2) counterfactually evaluate and compare alternative treatment regimens and their associated clinical outcomes. The first interactive feature allows the clinical provider to work together with the patient and take into account complex budget preferences, while the second interactive feature allows the clinical provider to combine their own preferences with the treatment recommendations, e.g. if the clinician favors a less aggressive approach, they could lower the doses in Diab-Rx's top treatment recommendation and simulate outcomes. The concept and implementation are both novel—as the computational tractability of Diab-Rx's search heuristic is important for implementing such interactive optimization wherein different cost weights need to be efficiently translated to optimal recommendations during a live consult.

It is believed that these interactive features could address one of the biggest barriers to collaborative decision making, by efficiently allowing patients to express preferences and guiding better customization of prescription to patient preferences while also optimizing outcomes. As such, the presently disclosed approach has the unique ability to generate recommendations that would accommodate mutual needs and preferences of both provider and patient for increased uptake, and to facilitate an efficient and evidence-driven shared decision-making process during a point-of-care consult.

Finally, the presently disclosed CDSS has a modular design to mitigate challenges due to lack of transportability or interoperability. It includes a data validation processor to mitigate the operational impact of real-world variations in data quality, data pre-processor for model training and inference that derive daily dosage information for patients' current and previous visits from raw EMR data, as well as a performance monitoring processor to ensure robustness during deployment. The whole set up is customizable for a variety of extensions as the optimization and interactive components can be parameterized with other important end-user considerations (e.g., side effect tolerance levels, safety override features), clinically applicable thresholds (e.g., for early stage or older patients), and/or integration requirements for different clinical settings, systems or workflows (e.g., beyond specialist outpatient consult to primary care or remote care settings).

### EXAMPLES

### Type 2 Diabetes CDSS Application Example

Significant advances have been made in the mechanistic understanding of diabetes treatment, drug mechanisms, and formulations. However, in clinical practice, maintaining patients within clinically prescribed target ranges remains highly challenging. During outpatient visits, physicians are routinely faced with challenges of choosing the optimal drug mix and dose to best cater to an individual patient's clinical condition and response profile.

As such, there is a need for treatment advisory tools that can enable targeted and personalized prescriptions with a view to optimizing clinical outcomes at price points that are accessible to cohorts across the socioeconomic strata. These needs are particularly relevant in translating healthcare beyond the acute hospital setting to the primary care setting.

The ideal treatment advisory strategies for type 2 diabetes (T2D) would leverage medical record data on the individual patient's evolving clinical history and response patterns to past prescriptions, consider expected impact on clinical outcomes of interest, and optimize the drug name and dose to provide the best likelihood of achieving desired targets. Furthermore, the advisory strategy would be developed with a view to account for clinical inertia due to cost and side-effect profiles, in tandem with the clinical outcome.

Here, an AI drug mix and dose advisory (Diab-Rx) to recommend pharmacological treatments for patients with type 2 diabetes is introduced. Specifically, a predict-then-optimize approach was adopted. First, given a patient's medical profile, machine learning was employed to model glycated hemoglobin (HbA1c) outcomes under different prescription regimens. Then, leveraging this prediction model, alongside clinical knowledge-based guidelines, a single-objective decision algorithm was developed to identify the minimal drug mix and dose changes required to best achieve the glycemic target. Finally, this decision algorithm was extended to address the more general multi-objective optimization problem of identifying drug mix and dose changes required to improve glycemic control, reduce costs, and increase the incidence of recommending preferred medications, all while fulfilling clinical knowledge-based guidelines.

### Overview of Approach and Task

### Methods Overview

Diab-Rx was developed and evaluated using electronic medical record (EMR) data from the SingHealth Diabetes Registry (SDR). A predict-then-optimize approach was employed for both the single objective and multi-objective versions. First, an HbA1c prediction model was developed to evaluate glycemic outcomes. The model was based on a decision-tree algorithm. Performance of the model was characterized both for prediction accuracy and causality in mapping prescription regimens to HbA1c outcomes. Second, a search heuristic was developed to identify the drug mix and dose regimen that optimizes the single glycemic control objective, while accounting for clinical knowledge-based guidelines. The value-add of including drug-dose specifics in the decision algorithm was assessed. Third, the search heuristic was expanded to identify drug mix and dose regimens that best balance the trade-off amongst the multiple objectives of improving HbA1c outcomes, lowering costs, and recommending preferred medications. The predicted impact of the multi-objective decision algorithm's recommendations on glycemic outcomes, costs, and the incidence of recommendation of preferred medications, was assessed for a large test set drawn from retrospective data.

### Dataset

The approach was developed and evaluated using anonymized electronic medical records from the SingHealth Diabetes Registry (SDR). The SDR is a multi-center registry comprising high quality anonymized electronic medical record (EMR) data, across the primary and hospital-based care continuum, for multi-ethnic diabetes patient cohorts in Singapore. The study was approved by the SingHealth Centralized Institutional Review Board with a waiver of informed consent.

All patients in the SDR during 2013-2019 were assessed for eligibility. Patients with a recorded history of type 1 or gestational diabetes were excluded. Other patients were included if they had outpatient visits with glycemic control prescriptions (‘prescription visits’) meeting the following criteria: (i) preceded by at least one prior prescription visit; (ii) preceded by at least one HbA1c measurement within 30 days before the visit; (iii) followed by at least one HbA1c measurement two to six months after the visit; and (iv) with no prescription change after the visit but before the next HbA1c measurement. Patients were split 80/20 into a ‘model development set’ for training and ‘test set’ for evaluation. The data splitting procedure was stratified by key demographic and clinical characteristics to ensure that the test set reflects the originating data distribution. While all visits were retained for patients in the model development set, only one visit was sampled per patient in the test set (to ensure patients with more visits are not over-represented in evaluations). The model development set consisted of 697 500 visits corresponding to 66 623 different patients, while the test set had data from 16 653 patients (visits).

### Data Preprocessing

For each patient in the development or evaluation sets, a set of 38 EMR variables were extracted, including demographics, physical measurements, laboratory values, medical history, and prescription details (see Table 1). For every prescription visit, a ‘visit medical profile’ was defined, said ‘visit medical profile’ comprising patient characteristics, pre-visit medical history, and previous visit prescription. For every prescription visit, an in-visit ‘treatment recommendation’ was defined, said in-visit ‘treatment recommendation’ represented as a prescribed set of generic name antidiabetic drugs (‘drug mix’), their daily dosages, and associated regimen ‘severity level’ (based on the number of oral antidiabetic drugs (OADs) and/or type of injectables). For every prescription visit, a ‘post-visit HbA1c’ outcome was defined, said ‘post-visit HbA1c’ outcome coded as the mean of all glycated hemoglobin (HbA1c) readings taken 2 to 6 months after the visit.

Feature representation details are as follows. Physical measurements and laboratory values were represented using the most recent measurements and the quartile summary statistics (25^{th}, 50^{th}, 75^{th} percentiles) of measurements taken within 1 year before the visit. The last HbA1c reading before the visit was denoted as ‘pre-visit HbA1c’. Diagnosis codes from any previous visits were mapped to features indicating past incidence of relevant comorbidities, diabetes complications, and contraindicating conditions. Previous surgery codes were used to infer past incidence of diabetes-related surgeries. Previous prescriptions for antihypertensive, lipid-lowering, and antiplatelet drugs were represented with indicator variables. The previous visit prescription was represented as the prescribed set of generic name antidiabetic drugs (drug mix) and their daily dosages. Drug mix was denoted as a subset of 31 generic name antidiabetic drugs (OADs, injectables, combination drugs) prescribed within the model development set, and was derived from raw electronic medical record data by mapping drug name information to generic name information. Daily dosage information was then derived by mapping drug frequency information to numerical information. Further, each prescription was associated with a regimen ‘severity level’ defined as: (1) 1 oral antidiabetic drug (OAD); (2) Multiple OAD; (3) First injectables (GLP-1 RA or long-acting insulin); (4) Premixed insulin; and (5) Rapid-acting insulin. The in-visit treatment recommendation was encoded as the change in drug mix, daily dosages, and severity level, relative to the previous prescription. An example of the input and output of this preprocessing routine is given in FIG. 5.

TABLE 1
Description of EMR data variables included in the development and evaluation
datasets.

| Category | Description |
|---|---|
| Demographics | Age (on visit date) |
| | Gender |
| | Ethnicity |
| | Housing type (patient-report of ownership, type, and size of housing) |
| Visit | Visit date |
| information | Institution |
| Physical | Measurement date |
| measurements | Blood pressure |
| | Body Mass index |
| Laboratory | Measurement date |
| data | Glycemic indices: Glycated hemoglobin (HbA_{1c}), Fasting glucose |
| | Renal function: Estimated glomerular filtration rate (eGFR, based on serum |
| | creatinine values per the 2009 CKD-EPI equation), Serum urea, Potassium |
| | serum |
| | Urinalysis: Urine microalbumin creatinine ratio, Urine protein creatinine ratio |
| | Lipid panel: Total cholesterol, Low-density lipoprotein cholesterol, High-density |
| | lipoprotein cholesterol, Triglycerides |
| | Liver function: Albumin, Aspartate aminotransferase, Gamma-glutamyl |
| | transferase, Alanine transaminase |
| | Blood counts: Hemoglobin, White blood cells, Platelets |
| | Other: Serum N-Terminal Pro-B-Type Natriuretic Peptide, Serum creatinine |
| | kinase |
| Medical | Past diagnosis or surgery codes (SNOMED, ICD, or Singapore Ministry of Health |
| history | Table of Surgical Procedures codes) |
| | Diabetes duration (based on diabetes diagnosis year reported) |
| | Smoking history (whether non-smoker, ex-smoker, smoker) |
| Prescriptions | Prescribed date, drug name, dosage, frequency for antidiabetic drugs |
| | Prescribed date for lipid-lowering, antihypertensive, and anti-platelet drugs |

### Treatment Recommendation Task

Given a patient's visit medical profile, the task is to recommend an appropriate drug mix and daily dose regimen while fulfilling clinical knowledge-based guidelines. For the single objective treatment optimization problem, the aim is to optimize glycemic control (defined as post-visit HbA1c at or under 7%). For the more general multi-objective treatment optimization problem, the aim is to improve glycemic control, lower costs, and recommend preferred medications.

Treatment options: The dataset contained outpatient prescriptions for 31 antidiabetic drugs (OADs, injectables, combination drugs). Of these, 6 drugs had very low outpatient utilization rates and were excluded from the treatment menu. Hence, the drug mix options were defined as all possible subsets of the remaining 25 generic name antidiabetic medications (Table 2). Cumulative daily dosage options were defined using local formulary configurations. Therefore, Diab-Rx's treatment options comprised of combinations of generic drug names and their daily dosages.

Clinical knowledge-based guidelines: To facilitate meaningful treatment recommendations, established clinical practice standards published by the American Diabetes Association (ADA), the European Association for the Study of Diabetes, and the Agency for Care Effectiveness, Singapore, drug label requirements from MIMS Singapore, and the experience of clinical experts in managing Type 2 diabetes were distilled into a knowledge-base for diabetes pharmacotherapy. In the exemplary implementation, the knowledge-base included 1) important medication contraindications (e.g., based on age, estimated glomerular filtration rate or eGFR, medical history), 2) restrictions on drug combinations (e.g., to restrict pairing of drugs such as sulphonylurea, meglitinide, premixed and/or rapid-acting insulins with known hypoglycemia risks; and pairing of dipeptidyl peptidase-4 (DPP-4) inhibitor and GLP-1 RA), 3) renal dosing requirements (e.g., in cases with eGFR<60 based on drug label requirements), as well as 4) prioritization of drugs (e.g., based on patient-specific factors and practice requirements, especially to prioritize indication-based treatments in patients with heart failure, cardiovascular disease, and/or kidney disease).). As many components of the knowledge-base depend on drug name and dose specifics, a decision algorithm with sufficiently granular representation of treatment options is needed to appropriately account for these clinical requirements.

TABLE 2
The list of treatment options available to the algorithm, based on the 31
generic name medications available in the dataset.

| | | | Can be |
|---|---|---|---|
| Type^{†} | Class | Generic name | recommended?* |
| OAD | Biguanides | Metformin | Y |
| OAD | Biguanides | Metformin extended release | Y |
| OAD | Sulphonylureas | Glibenclamide | Y |
| OAD | Sulphonylureas | Gliclazide | Y |
| OAD | Sulphonylureas | Gliclazide modified release | Y |
| OAD | Sulphonylureas | Glimepiride | Y |
| OAD | Sulphonylureas | Glipizide | Y |
| OAD | Sulphonylureas | Tolbutamide | Y |
| OAD | α-glucosidase | Acarbose | Y |
| | inhibitors | | |
| OAD | DPP-4 inhibitors | Linagliptin | Y |
| OAD | DPP-4 inhibitors | Saxagliptin | Y |
| OAD | DPP-4 inhibitors | Sitagliptin | Y |
| OAD | DPP-4 inhibitors | Vildagliptin | Y |
| OAD | SGLT2 inhibitors | Canagliflozin | Y |
| OAD | SGLT2 inhibitors | Dapagliflozin | Y |
| OAD | SGLT2 inhibitors | Empagliflozin | Y |
| OAD | Meglitinides | Nateglinide | N |
| OAD | Meglitinides | Repaglinide | N |
| OAD | Thiazolidinediones | Pioglitazone | N |
| INJ | GLP-1 RA | Liraglutide | Y |
| INJ | Long-acting | Insulin detemir | Y |
| | insulins | | |
| INJ | Long-acting | Insulin glargine | Y |
| | insulins | | |
| INJ | Intermediate- | Insulin isophane | N |
| | acting insulins | | |
| INJ | Premixed insulins | Insulin aspart insulin protamine aspart | Y |
| INJ | Premixed insulins | Insulin lispro protamine (75%) insulin | Y |
| | | lispro (25%) | |
| INJ | Premixed insulins | Insulin lispro protamine (50%) insulin | Y |
| | | lispro (50%) | |
| INJ | Premixed insulins | Insulin isophane insulin soluble (NPH) | N |
| INJ | Short-acting | Insulin soluble | N |
| | insulin | | |
| INJ | Rapid-acting | Insulin aspart | Y |
| | insulin | | |
| INJ | Rapid-acting | Insulin lispro | Y |
| | insulin | | |
| INJ | Rapid-acting | Insulin glulisine | Y |
| | insulin | | |

^{†}OAD: Oral Antidiabetic Drug; INJ: Injectable Antidiabetic Drug
*6 of 31 drugs excluded from the option list for treatment recommendation due to low outpatient utilization rates. Even if a drug cannot be prescribed, it can be continued if included in previous visit prescription.

### Proposed Solution

One instance of the proposed predict-then-optimize solution to address the above treatment recommendation task is presented in the following sections. First, a prediction model to evaluate glycemic outcomes as a function of drug mix and dose regimens is described. Performance characterizations to show viability of this prediction task are provided. Second, a search heuristic to identify the drug mix and dose regimen that optimizes the glycemic control objective is introduced. A demonstration of the value of accounting for drug and dose specifics within this heuristic is included. Third, the search heuristic is expanded to address the more general task of concomitantly optimizing multiple objectives including glycemic outcomes, cost and prioritization of preferred regimens. A characterization of the impact of recommendations from this expanded heuristic on each of the objectives, relative to standard of care (SoC) prescriptions, is provided.

A prediction model to evaluate glycemic outcomes

The modelling task is to predict the post-visit HbA1c given the visit medical profile and a plausible in-visit treatment recommendation. For this task, eXtreme Gradient Boosting (XGBoost), a tree-based ensemble machine learning algorithm, was used as it offers good computational efficiency for training and inference. The visit medical profile representation was condensed via feature selection with the Least Absolute Shrinkage and Selection Operator (LASSO). The in-visit treatment recommendation was represented using the complete drug mix and dose regimen feature set. The hyperparameters (learning rate and number of estimators) were tuned using 4-fold cross-validation and grid search across 5 learning rate settings {0.001, 0.005, 0.01, 0.05, 0.1} and 3 number of estimators settings {500, 1000, 1500}. The model with the highest R² was chosen as Diab-Rx's outcome prediction model. Model performance for both regression and classification were evaluated in relation to ground truth post-visit glycemic outcomes under standard of care (SoC) prescriptions.

The performance of the HbA1c prediction model was evaluated in relation to observed post-visit glycemic outcomes under standard of care (SoC) prescriptions. Causality checks were also performed to determine whether predictions corresponding to counterfactual prescription regimens were sensible. For each generic name medication, one of the following prescription changes relative to each patient's previous visit prescription recommendation was applied to each visit: 1) Increase dose to the maximum possible value, 2) Increase dose to half the maximum possible value, 3) Add at the minimum dose, 4) Add at the maximum dose, 5) Drop. The HbA1c prediction model was used to predict the HbA_{1c} outcome corresponding to no regimen change, and also to predict the HbA_{1c} outcome corresponding to the regimen change of interest. If the direction of change between the two values was as expected—i.e., if dose increases or medication additions led to a lower predicted HbA_{1c} outcome, and if dropping medications led to a higher predicted HbA_{1c} outcome—the model was rated as performing sensibly. The rates of sensible predictions for each possible action were then calculated.

On the test set, the XGBoost model achieved AUC of 0.79 for classification of whether post-visit HbA1c is within glycemic control target; and R² of 0.57 for post-visit HbA1c prediction, respectively (Table 3). The most important features, based on the average performance gain, pertained to the pre-visit HbA1c (the last HbA1c reading before the visit), and previous and current visit prescription information (Table 4). Since the prediction model is also required to learn treatment effects, and to accurately predict HbA_{1c} outcomes corresponding to counterfactual choices of the prescription regimen, the sensibility of the predictions corresponding to counterfactual prescription regimens was assessed. The results (Table 5) show that the prediction model makes sensible forecasts in the majority of cases, suggesting that it is able to learn treatment effects reasonably well.

TABLE 3
Performance of HbA1c prediction model on the test set.

| | | | | RMSE^{‡} | |
|---|---|---|---|---|---|
| Set of visits | MAPE* | MDAPE^{†} | R² | (%) | AUC^{§} |
| All visits | 7.65 | 5.30 | 0.57 | 0.89 | 0.791 |
| Visits with | 9.61 | 7.01 | 0.54 | 1.12 | 0.754 |
| prescription | | | | | |
| change | | | | | |
| under Soc^{∥} | | | | | |
| Visits | 6.68 | 4.69 | 0.56 | 0.75 | 0.794 |
| without | | | | | |
| prescription | | | | | |
| change | | | | | |
| under SoC | | | | | |

*Mean Absolute Percentage Error
^{†}Median Absolute Percentage Error
^{‡}Root Mean Square Error
^{§}Area under the receiving operating characteristic (ROC) Curve
^{∥}Change in either drug mix or dose relative to the previous visit prescription

TABLE 4
The list of 15 most important features for the HbA_{1c} prediction model used by
Diab-Rx, based on feature importance scores calculated using the XGBoost Python module.
Features are listed in descending order of impact on model performance.

| | Feature |
|---|---|
| 1 | Pre-visit HbA_{1c} |
| 2 | Previous visit regimen severity level |
| 3 | 75^{th} percentile of HbA_{1c} measurements within past year |
| 4 | Change in regimen severity level (current vs. previous visit) |
| 5 | Diabetes duration |
| 6 | 50^{th} percentile of HbA_{1c} measurements within past year |
| 7 | Change in daily dose of metformin between current and previous visits |
| 8 | 25^{th} percentile of HbA_{1c} measurements within past year |
| 9 | Previous visit glipizide dose |
| 10 | Previous visit dapagliflozin dose |
| 11 | Change in daily dose of dapagliflozin between current and previous visits |
| 12 | Most recent fasting glucose measurement |
| 13 | Previous visit metformin dose |
| 14 | Visit setting (primary/specialist care) |
| 15 | Change in daily dose of glipizide between current and previous visits |

TABLE 5
Results of causality check. For each generic name medication, one of the
five actions was applied to all visits where the action is applicable. For each visit, the causality
check is considered to pass if the direction of the predicted blood glucose level change is as
expected. N.A. indicates that there are no visits where the associated action is applicable.

| | Fraction of visits for which causality check passes | | | | |
|---|---|---|---|---|---|
| | Incr dose | Incr dose | Add at min | Add at max | |
| Generic name | to max | to 1/2 max | dose | dose | Drop |
| glibenclamide | 0.73 | 0.37 | 0.62 | 0.62 | 0.97 |
| gliclazide | 0.55 | 0.82 | 0.98 | 0.98 | 0.99 |
| gliclazide mr | 0.86 | 0.98 | 0.98 | 0.50 | 0.99 |
| glimepiride | 0.88 | 0.74 | 0.90 | 0.90 | 0.86 |
| glipizide | 0.83 | 0.86 | 0.97 | 0.88 | 0.99 |
| tolbutamide | 0.96 | 0.69 | 0.96 | 0.96 | 0.98 |
| dapagliflozin | 0.98 | 0.81 | 0.92 | 0.92 | 0.98 |
| empagliflozin | 0.87 | 0.85 | 0.85 | 0.86 | 0.43 |
| canagliflozin | 1.00 | 1.00 | 0.84 | 0.84 | 0.90 |
| metformin | 0.97 | 0.92 | 0.82 | 0.67 | 0.98 |
| metformin xr | 0.98 | 0.99 | 0.89 | 0.93 | 0.36 |
| linagliptin | N.A. | N.A. | 0.99 | 0.99 | 0.99 |
| sitagliptin | 0.95 | 0.95 | 0.98 | 0.99 | 0.97 |
| vildagliptin | 0.98 | 0.00 | 0.96 | 0.96 | 0.93 |
| saxagliptin | 0.04 | 0.04 | 0.33 | 0.81 | 0.89 |
| acarbose | 0.97 | 0.89 | 0.82 | 0.86 | 0.90 |
| insulin glulisine | 0.94 | 0.94 | 0.66 | 0.86 | 0.46 |
| insulin aspart | 0.43 | 0.31 | 0.66 | 0.71 | 0.83 |
| insulin lispro | 1.00 | 1.00 | 0.66 | 0.94 | — |
| insulin aspart protamine | 0.54 | 0.80 | 0.67 | 0.78 | 0.32 |
| insulin aspart | | | | | |
| insulin lispro protamine 75 | 0.71 | 0.50 | 0.67 | 0.70 | 0.67 |
| insulin lispro 25 | | | | | |
| insulin lispro protamine 50 | N.A. | N.A. | 0.67 | 0.88 | — |
| insulin lispro 50 | | | | | |
| insulin detemir | 0.02 | 0.02 | 0.77 | 0.38 | 0.22 |
| insulin glargine | 0.77 | 0.69 | 0.77 | 0.83 | 0.62 |
| liraglutide | 0.94 | 0.98 | 0.93 | 0.91 | 0.78 |

### a Search Heuristic for the Single Objective Treatment Optimization Task

Single glycemic objective optimization: To generate drug mix and dose recommendations for the single glycemic objective optimization problem, a novel and intuitive search scheme was designed. This scheme leverages the knowledge-base and the predicted post-visit glycemic outcomes; and searches for the regimen that is expected to bring the patient into or as close to glycemic control as possible, while attempting to minimize the degree of intensification.

The search starts with the patient's previous prescription regimen and considers changes stepwise in order of increasing intensity. First, Diab-Rx drops medications and lowers doses as required by medication safety guidelines. If the resulting regimen is predicted to be able to achieve glycemic control (as per the HbA1c prediction model), no further change is recommended. Otherwise, Diab-Rx checks whether the patient can be brought into glycemic control by increasing dose of an existing medication, subject to dosing guidelines. If this is insufficient, substitution of up to one new medication is attempted. If this is also insufficient, Diab-Rx tries to add up to one new medication. To choose the new medication, Diab-Rx constructs a list of feasible options, caps severity level changes to one if pre-visit HbA_{1c} is under 8% and to two otherwise, and prioritizes the options as per knowledge-based guidelines. Then, Diab-Rx searches the list to select the highest priority option predicted to bring the patient into glycemic control, and recommends the minimum dose required for glycemic control. If glycemic control is not predicted to be possible, Diab-Rx recommends the regimen with the lowest post-visit HbA_{1c}. Notably, the efficiency and accuracy of the search is enhanced with careful consideration of dose effects based on properties described in embodiments above.

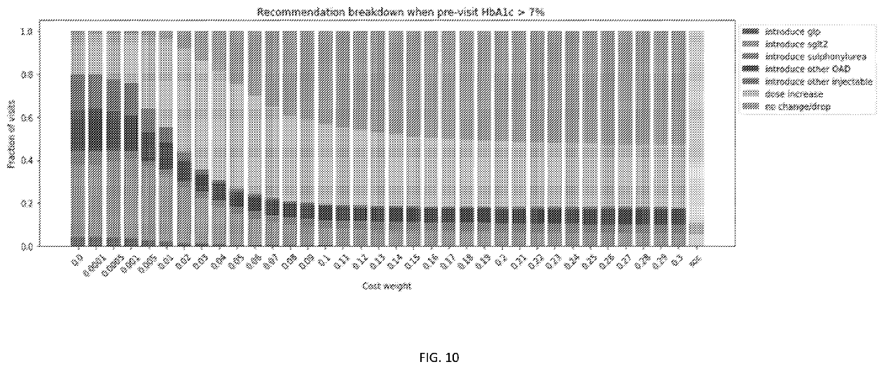

Estimation of HbA1c benefit: Potential post-visit HbA1c outcomes under Diab-Rx were compared with the observed post-visit HbA1c outcomes under the Standard of Care (SoC). As the evaluations were on retrospective data, and counterfactuals were not available, the HbA_{1c} prediction model was used to estimate potential post-visit HbA_{1c} outcomes resulting from alternative prescription recommendations. Then, the HbA_{1c} benefits relative to the SoC were estimated by taking the difference between the predicted HbA1c outcomes under the optimizer's recommendations and the observed HbA_{1c} outcome under the SoC recommendation. As shown in FIG. 8, the absolute post-visit HbA1c outcomes expected under recommendations from Diab-Rx were concentrated heavily around the glycemic control target of 7%, while outcomes under SoC were more right-skewed. This is indicative of Diab-Rx recommending intensification of treatment to just the extent required for glycemic control, unlike SoC which exhibits substantial inertia to intensify treatment. In particular, for test set visits where Diab-Rx recommends a prescription change, the mean (±SE) post-visit HbA1c benefit expected under Diab-Rx, relative to SoC, was 0.36±0.01% (P<0.0001, one-sided paired sample Student's t-test). Benefits were higher for cases with higher pre-visit HbA1c.

Qualitative evaluation of Diab-Rx recommendations: Case reviews were conducted with a panel of practicing endocrinologists to assess the quality of Diab-Rx treatment recommendations and the value-add of optimizing over drug and dose options.

Specifically, 30 cases were sampled to represent diverse profiles across age bands, care settings, pre-visit HbA1c ranges, eGFR ranges, and previous visit regimen severity levels. For each case, recommendations were generated using Diab-Rx and two baseline approaches that omit drug name and/or dose considerations. For the first baseline, a previously proposed Drug Class-combination Advisor (DCA) that recommends categorical combinations of glucose-lowering drug classes (namely metformin, non-metformin oral agents, and/or injectables) was implemented (as per the original implementation, but excluding ‘no regimen’ from the treatment menu). For the second baseline, a Drug Mix Advisor (DMA) that recommends a combination of generic drug names was constructed. DMA was constructed to be identical to Diab-Rx in all aspects except in that it does not use dose information for prediction and optimization. Both the baselines leveraged HbA1c prediction models but with features represented to reflect the different granularity of treatment options (i.e., drug class combinations, drug name combinations instead of drug name and dose combinations). Further, each baseline incorporated all guidelines within the knowledge-base that were permitted by its medication representation.

Evaluators independently reviewed each case and provided their expert opinion on the extent to which Diab-Rx's recommendation is sensible (i.e., consistent with established clinical practice norms) and precise (i.e., provides sufficient specifics to resolve a clinical decision on drug mix and dose); and on how Diab-Rx's recommendation compares with recommendations from the two baselines. The reviewer's free-text comments were analyzed and coded by two research analysts. In cases where evaluators had differing opinions, consensus opinion of the panel was obtained via adjudication. For comparisons between Diab-Rx and the baselines, emergent themes on the key advantages and disadvantages were identified and discussed with the evaluation panel for consensus.

Diab-Rx was rated as clinically sensible in 17 of 30 cases, acceptable in 11 of 30 cases, and not sensible in 2 of 30 cases; and as sufficiently precise in 28 of 30 cases and somewhat precise in 2 of 30 cases. In the comparative assessment, Diab-Rx was generally rated as clinically more sensible and more precise than the baselines (Table 6). In cases where evaluators favored Diab-Rx over DMA, two main advantages for Diab-Rx emerged. The first advantage stemmed from the ability to appropriately recommend dose intensification to enhance glycemic control while avoiding new medications. The second advantage stemmed from the ability to prioritize medications per guidelines. Furthermore, as Diab-Rx recommendations included specifics on drug name and dose choices, evaluators found them to be less ambiguous and more informative than the baseline approaches, suggesting potential for higher clinical utility. Taken together, these results demonstrate how Diab-Rx's ability to account for drug-dose specifics could enhance the clinical relevance of the treatment recommendations.

TABLE 6
Results from comparative assessment of recommendations from Diab-Rx and
baseline approaches (DCA, DMA). Counts indicate the number of cases where Diab-Rx's
recommendation was rated as more, similarly, or less sensible or precise than the baseline
recommendation. Case counts are based on consensus ratings by the evaluation panel for
the 30 cases reviewed.

| | Evaluation Panel | Drug Class-combination | |
|---|---|---|---|
| Metric | Consensus | Advisor (DCA) | Drug Mix Advisor (DMA) |
| How | Diab-Rx is more sensible | 19 | 9 |
| sensible | Diab-Rx is similarly | 10 | 16 |
| | sensible | | |
| | Diab-Rx is less sensible | 1 | 5 |
| How | Diab-Rx is more precise | 30 | 19 |
| precise | Diab-Rx is similarly | 0 | 11^{∥} |
| | precise | | |
| | Diab-Rx is less precise | 0 | 0 |

### a Search Heuristic for the Multi-Objective Treatment Optimization Task

Multi-objective optimization: To generate recommendations for the multi-objective optimization problem, Diab-Rx employs a novel search heuristic that harnesses both the HbA1c prediction model and the knowledge-base to make a recommendation that balances the trade-off among maximizing the HbA_{1c} reduction, lowering costs, and recommending preferred medications given weights that control the trade-off among these objectives. More specifically, the heuristic solves for the treatment regimen that optimizes the Value Equation (2). The heuristic first generates the list of all possible options corresponding to adding one new medication, substituting one new medication, or increasing the dose of an existing medication. The heuristic then uses a novel dose tuning subroutine to tune the dose of new medications that are being added, or of existing medications for which the dose is being increased. This subroutine uses two consecutive binary search procedures to improve the computational tractability of the search compared to brute-force searching the set of possible doses. The heuristic is described in full in Algorithm 1 above.

Evaluation of trade-off among different objectives: Diab-Rx recommendations and SoC recommendations were compared in terms of HbA_{1c} outcomes, costs, and incidence of recommendation of preferred medications. Specifically, (1) the mean HbA_{1c} benefit relative to the SoC, (2) the mean monthly cost difference relative to the SoC, and (3) the mean difference in the rate of recommendation of the preferred medications relative to the SoC were estimated for different values of the hyperparameters controlling the tradeoff among the different objectives (Equation (2). For these evaluations, the medication preferences function in Equation (2), PREF, was set as the indicator function for whether SGLT2-i is prescribed when the patient has been diagnosed with heart failure or chronic kidney disease. Additionally, the dependence of the recommendations on the choice of hyperparameters was characterized.

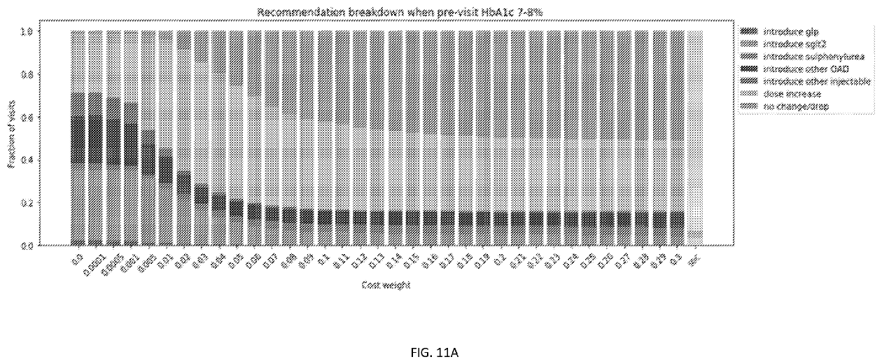

The results on the test set are given in FIGS. 9A and 9B. As expected, both the HbA_{1c} benefit and the mean cost difference are decreasing as the cost weight w₀ increases (FIG. 9A), and the HbA_{1c} benefit decreases while the rate of prescription of preferred medications increases as w₁ increases (FIG. 9B). Further, it is observed that for a cost weight of 0 (i.e., the optimizer completely disregards cost considerations), the recommended treatment regimen is on average almost $50/month more expensive than the SoC recommendation. By increasing the cost weight slightly, the mean cost of the recommended treatment regimen drops sharply. It is possible to achieve a mean HbA_{1c} benefit of around 0.125% over the SoC without increasing the mean monthly cost difference relative to the SoC. In fact, it is possible to continue to achieve a mean HbA_{1c} benefit over the SoC while incurring lower costs than the SoC. Similarly, when w₁ is 0 (i.e., the optimizer completely disregards medication preferences), the rate of SGLT2-i prescription by the optimizer is only slightly higher than the SoC. This rate increases sharply as w₁ is increased, while the mean HbA_{1c} benefit drops more slowly. These results demonstrate the significance of incorporating multifactorial considerations in the treatment recommendation problem, as they show that ignoring these considerations can lead to significantly more expensive recommendations with low incidence of recommendation of preferred medications.

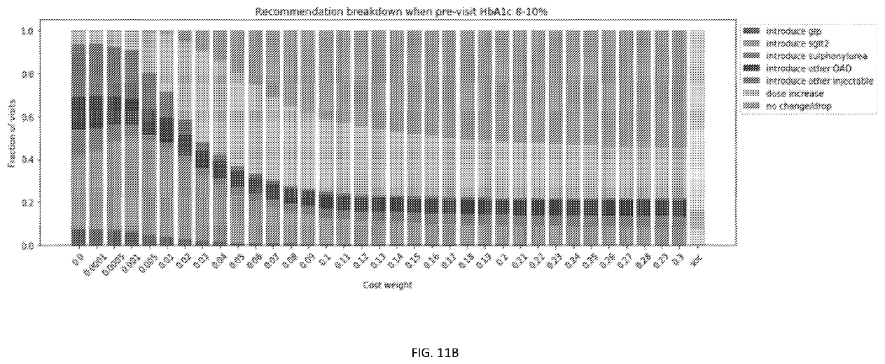

Additionally, the study qualitatively characterized the optimizer recommendations to examine how these recommendations change as the hyperparameters are varied. FIG. 10 plots the breakdown of the optimizer recommendations across patients with pre-visit HbA_{1c} greater than 7% as the hyperparameter w₀ is varied, where recommendations are grouped according to the introduction (either addition or substitution) of different drug subgroups and classes, dose increases, or no change/dropping an existing medication. As w₀ is increased, the no change option becomes more prevalent, but so does the dose increase option, and the introduction of SGLT2-i.

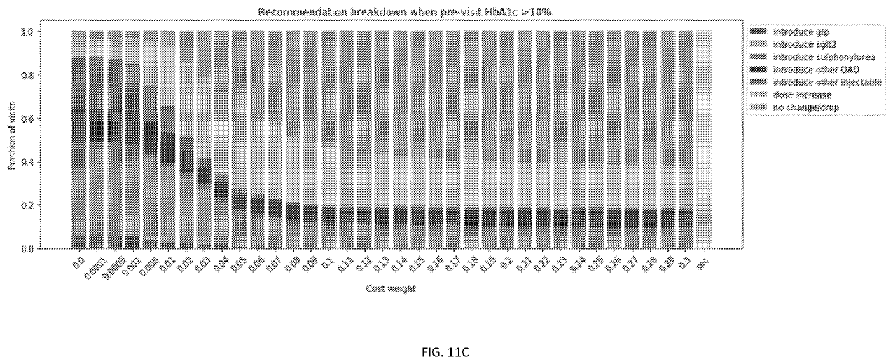

FIG. 11A to 11C plot the breakdown of recommendations stratified according to the patient pre-visit HbA_{1c} and show that this effect of increasing prevalence of SGLT2-i introduction is more pronounced for patients with higher pre-visit HbA_{1c}. Meanwhile, the rate of recommendation of GLP1-RA (a newer, more expensive injectable drug) decreases as w₀ increases. This shows that the optimizer is not simply saving costs by refraining from making recommendations but is in fact finding cheaper alternatives that can simultaneously manage costs as well as HbA_{1c} reductions. However, as w₀ continues to increase, the primary change observed is indeed an increase in prevalence of the no change or drop option, and the optimizer recommendation converges to the SoC (depicted on the extreme right of FIG. 10 and FIG. 11A to 11C).

In summary, the examples provided illustrate the application of the disclosed framework to the task of generating drug mix and dose recommendations for optimal glycemic management in type 2 diabetes. Further, they demonstrate the viability and value of applying the presently disclosed framework to a real-world clinical use case.

### Products and Applications

The method and system disclosed supports the practical translation of treatment recommendation approaches to clinical decision support. Although the study demonstrated the presently disclosed framework for a type 2 diabetes treatment recommendation use case in a clinical setting, one or more components of the presently disclosed approach can be generalized to other care settings, treatment advisory problem formulations, and disease conditions. Examples of other products and applications are provided in Table 7.

TABLE 7
Examples of products and applications of the presently disclosed method and
system.

| No | Disease | Use Case | Objective | Data Input | Decision Output |
|---|---|---|---|---|---|
| 1 | T2DM | Drug Class/ | Assist clinician at | EMR time | Generic drug name |
| | | Drug Mix/ | primary care or | series | and daily dosages, |
| | | Drug Mix & | specialist outpatient | | across one or |
| | | Dose | care setting to | | more drugs as |
| | | Recommendation | optimize overall | | needed |
| | | | prescription (glycemic | | |
| | | | control, disease | | |
| | | | progression) | | |
| 2 | T2DM | Insulin | Overcome uncertainty | EMR time | Transition from |
| | | Initiation | and inertia with | series | insulin-naïve state |
| | | | respect to when to | | to an initial insulin |
| | | | initiate patients on | | prescription |
| | | | insulin | | |
| 3 | T2DM | Insulin | Manage insulin based | Streaming | Optimal insulin |
| | | Titration | on patient needs, | home-based | drug names and |
| | | | tolerance levels, and | glucose | dosages |
| | | | other routine lifestyle | monitoring | |
| | | | factors (glycemic | data (e.g., | |
| | | | control, hypoglycemia, | from CGMs, | |
| | | | progression) | SMBG, Insulin | |
| | | | | Pumps) | |
| | | | | Optional: diet | |
| | | | | & exercise | |
| | | | | data | |
| 4 | T2DM | Insulin | Same as #3, but in | Streaming | Similar to #3, but |
| | | Titration | context of overall | device data as | also considering |
| | | | combination therapy | in #3 + EMR | the patient's |
| | | | (glycemic control, | data | overall prescription |
| | | | hypoglycemia, | | |
| | | | progression) | | |
| 5 | HTN | Blood | Assist clinician at | EMR time | Generic drug name |
| | | Pressure | primary care or | series | and daily dosages, |
| | | Medication | specialist outpatient | | across one or |
| | | Recommendation | care setting to | | more drugs as |
| | | | optimize overall | | needed |
| | | | prescription (blood | | |
| | | | pressure control, | | |
| | | | disease progression) | | |
| 6 | HLD | Cholesterol | Assist clinician at | EMR time | Generic drug name |
| | | Medication | primary care or | series | and daily dosages, |
| | | Recommendation | specialist outpatient | | across one or |
| | | | care setting to | | more drugs as |
| | | | optimize overall | | needed |
| | | | prescription (control of | | |
| | | | cholesterol levels, | | |
| | | | disease progression) | | |
| 7 | T2DM, | Medication | Assist clinician at | EMR time | Generic drug name |
| | HTN, HLD | Recommendation | primary care or | series | and daily dosages, |
| | | for Multiple | specialist outpatient | | across one or |
| | | Cardiometabolic | care settings to | | more drugs as |
| | | Conditions | optimize overall | | needed |
| | | | prescription for any | | |
| | | | combinations of | | |
| | | | common chronic | | |
| | | | cardiometabolic | | |
| | | | conditions | | |
| 8 | Other | Oncology | Clinical decision | EMR | Drug mix and |
| | Disease | Pain | support | | dose, for pumps, |
| | | Medication | Shared decision | | combination |
| | | Dosing | making | | therapies, etc |
| | | Systems | | | |

*Demonstrator with Diab-Rx corresponds to #1
*T2DM: Type 2 Diabetes Mellitus; T1DM: Type 1 Diabetes Mellitus; HTN: Hypertension; HLD: Hyperlipidemia
*CGM (continuous glucose monitors), SMBG (self-monitoring of blood glucose)

Beyond the objectives listed, the proposed framework could also be used for related objectives. A non-exhaustive list of possibilities is below:
1. Generate possible decisions that could optimize trade-offs for clinical care (e.g., cost vs. multifactorial risk profiles vs. outcomes).
2. Set care management targets given prescription and incentivize allied health teams to work with patients on lifestyle modifications to achieve these targets.
3. Stratify patients into high vs. low clinical risk categories for focused care management.
4. Personalized assessment of plausible decisions on outcomes of interest.

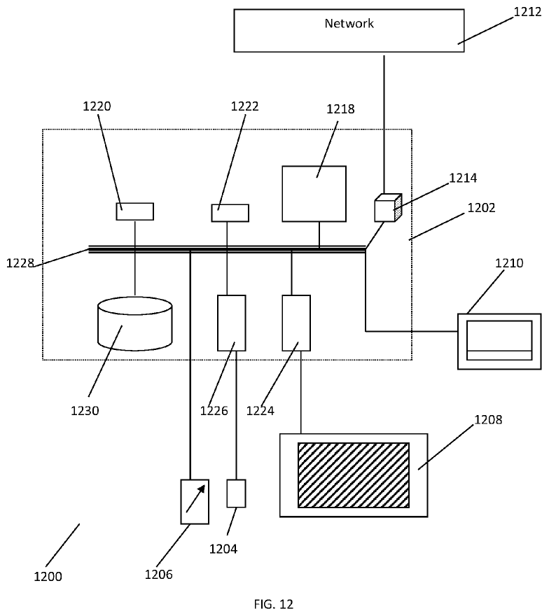

Different example embodiments can be implemented in the context of data structure, program modules, program and computer instructions executed in a computer implemented environment. A general purpose computing environment is briefly disclosed herein. One or more example embodiments may be embodied in one or more computer systems, such as is schematically illustrated in FIG. 12.

### Computing Device

One or more example embodiments may be implemented as software, such as a computer program being executed within a computer system 1200, and instructing the computer system 1200 to conduct a method of an example embodiment. Examples of a computer system may include a personal computer, desktop computer laptop, server computer, a computing node within a cluster and the like.

The computer system 1200 comprises a computer unit 1202, input modules such as a keyboard 1204 and a pointing device 1206 and a plurality of output devices such as a display 1208, and printer 1210. A user can interact with the computer unit 1202 using the above devices. The pointing device can be implemented with a mouse, track ball, pen device or any similar device. One or more other input devices (not shown) such as a joystick, game pad, satellite dish, scanner, touch sensitive screen or the like can also be connected to the computer unit 1202. The display 1208 may include a cathode ray tube (CRT), liquid crystal display (LCD), field emission display (FED), plasma display or any other device that produces an image that is viewable by the user.

The computer unit 1202 can be connected to a computer network 1212 via a suitable transceiver device 1214, to enable access to e.g. the Internet or other network systems such as Local Area Network (LAN) or Wide Area Network (WAN) or a personal network. The network 1212 can comprise a server, a router, a network personal computer, a peer device or other common network node, a wireless telephone or wireless personal digital assistant. Networking environments may be found in offices, enterprise-wide computer networks and home computer systems etc. The transceiver device 1214 can be a modem/router unit located within or external to the computer unit 1202, and may be any type of modem/router such as a cable modem or a satellite modem.

It will be appreciated that network connections shown are exemplary and other ways of establishing a communications link between computers can be used. The existence of any of various protocols, such as TCP/IP, Frame Relay, Ethernet, FTP, HTTP and the like, is presumed, and the computer unit 1202 can be operated in a client-server configuration to permit a user to retrieve web pages from a web-based server. Furthermore, any of various web browsers can be used to display and manipulate data on web pages.

The computer unit 1202 in the example comprises a processor 1218, a Random Access Memory (RAM) 1220 and a Read Only Memory (ROM) 1222. The ROM 1222 can be a system memory storing basic input/output system (BIOS) information. The RAM 1220 can store one or more program modules such as operating systems, application programs and program data.

The computer unit 1202 further comprises a number of Input/Output (I/O) interface units, for example I/O interface unit 1224 to the display 1208, and I/O interface unit 1226 to the keyboard 1204. The components of the computer unit 1202 typically communicate and interface/couple connectedly via an interconnected system bus 1228 and in a manner known to the person skilled in the relevant art. The bus 1228 can be any of several types of bus structures including a memory bus or memory controller, a peripheral bus, and a local bus using any of a variety of bus architectures.

It will be appreciated that other devices can also be connected to the system bus 1228. For example, a universal serial bus (USB) interface can be used for coupling a video or digital camera to the system bus 1228. An IEEE 1394 interface may be used to couple additional devices to the computer unit 1202. Other manufacturer interfaces are also possible such as FireWire developed by Apple Computer and i.Link developed by Sony. Coupling of devices to the system bus 1228 can also be via a parallel port, a game port, a PCI board or any other interface used to couple an input device to a computer. It will also be appreciated that, while the components are not shown in the figure, sound/audio can be recorded and reproduced with a microphone and a speaker. A sound card may be used to couple a microphone and a speaker to the system bus 1228. It will be appreciated that several peripheral devices can be coupled to the system bus 1228 via alternative interfaces simultaneously.

An application program can be supplied to the user of the computer system 1200 being encoded/stored on a data storage medium such as a CD-ROM or flash memory carrier. The application program can be read using a corresponding data storage medium drive of a data storage device 1230. The data storage medium is not limited to being portable and can include instances of being embedded in the computer unit 1202. The data storage device 1230 can comprise a hard disk interface unit and/or a removable memory interface unit (both not shown in detail) respectively coupling a hard disk drive and/or a removable memory drive to the system bus 1228. This can enable reading/writing of data. Examples of removable memory drives include magnetic disk drives and optical disk drives. The drives and their associated computer-readable media, such as a floppy disk provide nonvolatile storage of computer readable instructions, data structures, program modules and other data for the computer unit 1202. It will be appreciated that the computer unit 1202 may include several of such drives. Furthermore, the computer unit 1202 may include drives for interfacing with other types of computer readable media.

The application program is read and controlled in its execution by the processor 1218. Intermediate storage of program data may be accomplished using RAM 1220. The method(s) of the example embodiments can be implemented as computer readable instructions, computer executable components, or software modules. One or more software modules may alternatively be used. These can include an executable program, a data link library, a configuration file, a database, a graphical image, a binary data file, a text data file, an object file, a source code file, or the like. When one or more computer processors execute one or more of the software modules, the software modules interact to cause one or more computer systems to perform according to the teachings herein.

The operation of the computer unit 1202 can be controlled by a variety of different program modules. Examples of program modules are routines, programs, objects, components, data structures, libraries, etc. that perform particular tasks or implement particular abstract data types. The example embodiments may also be practiced with other computer system configurations, including handheld devices, multiprocessor systems, microprocessor-based or programmable consumer electronics, network PCs, minicomputers, mainframe computers, personal digital assistants, mobile telephones and the like. Furthermore, the example embodiments may also be practiced in distributed computing environments where tasks are performed by remote processing devices that are linked through a wireless or wired communications network. In a distributed computing environment, program modules may be located in both local and remote memory storage devices.

The example embodiments may also be practiced with other computer system configurations, including handheld devices, multiprocessor systems/servers, microprocessor-based or programmable consumer electronics, network PCs, minicomputers, mainframe computers, personal digital assistants, mobile telephones, patient device/sensor and the like. Furthermore, the example embodiments may also be practiced in distributed computing environments where tasks are performed by remote processing devices that are linked through a wireless or wired communications network. In a distributed computing environment, program modules may be located in both local and remote memory storage devices.

### Distributed Computing Environment

Various example embodiments of the presently disclosed method and system may be implemented in distributed computing system environments where local and remote computer systems both perform tasks. In various embodiments, the local and remote computer systems are linked (either by hardwired data links, wireless data links, or by a combination of hardwired and wireless data links) through a network. In various embodiments, one or more processors for implementing the methods disclosed herein may be situated in a single geographic location (e.g., within a home environment, an office environment, or a server farm). In various embodiments, one or more processors for implementing the methods as disclosed herein may be distributed across a plurality of geographic locations. In various embodiments, in a distributed computing system environment, program modules may be located in both local and remote memory storage devices.

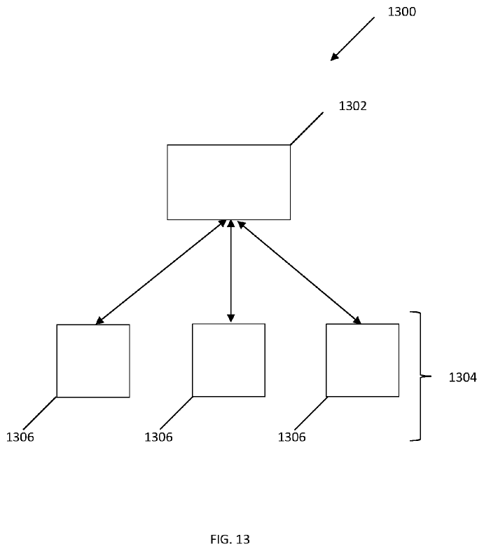

FIG. 13 illustrates an example of a distributed computing system environment 1300. The distributed computing system environment 1300 comprises a control server 1302 and at least one distributed pool 1304 of computing resources connected to the control server 1302 via communications network. The distributed computing system environment 1300 may further comprise additional distributed pools. The computing resources within the distributed pool 1304 may be dedicated for exclusive use within the distributed pool 1304 or shared with other additional distributed pools within the distributed processing system and with external applications. In addition, the computing resources in the distributed pool may be dynamically allocated, allowing computing devices 1306 to be added to or removed from the distributed pool 1304 as needed.

In various embodiments, the control server 1302 functions as a software application responsible for overseeing and managing the computing devices 1306 within the distributed pool 1304. The control server 1302 may be implemented on a computing device, such as the one described above in reference to FIG. 12. In various embodiments, the control server 1302 and computing devices 1306 in the distributed pool 1304 may communicate through an application programming interface (API), e.g., a Web services API. In various embodiments, the control server 1302 may be configured to provide users administration and computing resource management functionalities, allowing the control server 1302 to control the distributed pool 1304, e.g., by defining resource availability, submitting, monitoring, and controlling tasks performed by computing devices 1306, determining task priorities, or managing the storage and transmission of data resulting from completed tasks.

In various embodiments, the control server 1302 may be configured to identify a computing task slated for execution across the distributed computing system environment 1300. The computing task may be segmented into multiple work units that are capable of being executed by the different computing devices 1306 within the distributed pool 1304. The subdivision and concurrent execution of the computing task across the computing devices 1306 enable efficient parallel processing, leading to enhanced task completion performance (e.g., quicker execution, minimized resource consumption) compared to a non-distributed computing system environment.

In various embodiments, the computing devices 1306 in the distributed pool 1304 may have different configurations tailored for optimal performance in their specific roles. For instance, one subset of computing devices 1306 may be exclusively designated for collecting and/or pre-processing patient data, while another subset may be dedicated to training machine learning models. The first subset may possess fewer random-access memory (RAM) and/or processors compared to the second subset, considering the potential requirement for greater resources during the training of machine learning models.

In various embodiments, the computing devices 1306 within the distributed pool 1304 may simultaneously execute their assigned tasks and, upon completion, store the results in a storage device or transmit them back to the control server 1302. The control server 1302 may compile the results or, if necessary, redistribute them to the respective computing devices 1306 for further processing.

In various embodiments, the distributed computing system environment 1300 may be implemented within a cloud computing framework. In this context, “cloud computing” refers to a model that facilitates on-demand network access to a shared and configurable set of computing resources. For example, communication between the control server 1302 and the computing devices 1306 in the distributed pool 1304 may occur through the cloud. In various embodiments therefore, the control server 1302 and computing devices 1306 may be in geographically different locations. Cloud computing may be utilized to provide on-demand access to configurable computing resources that can be rapidly provisioned, virtualized, and released with minimal management effort. The cloud-computing model may exhibit characteristics such as on-demand self-service, broad network access, resource pooling, rapid elasticity, measured service, and more. Additionally, various service models like Software as a Service (SaaS), Platform as a Service (PaaS), and Infrastructure as a Service (IaaS), as well as deployment models such as private cloud, community cloud, public cloud, hybrid cloud, are encompassed within the present disclosure when referring to a “cloud-computing environment.”

### Applications

In the described example embodiments, the method and system may advantageously embed multi-objective optimization capability to generate and compare multiple granular recommendations that nearly optimize target outcomes but do so by favouring different trade-offs (e.g., cost, practicability).

In the described example embodiments, the method and system may advantageously provide users the ability to interactively explore efficacy of alternative regimens that may suit clinician or patient preferences or tolerance levels.

In the described example embodiments, the method and system may advantageously feature a modular and robust decision support system design that is highly customizable for varying input data quality levels, end-user considerations, and/or integration requirements associated with distinct clinical use cases, settings, systems, and workflows.

Collectively, these features serve to bring data-driven evidence and recommendations to the point-of-care in a manner that provides flexibility, considers practical factors such as those driving therapeutic inertia, and facilitates a shared decision-making process.

It will be appreciated by a person skilled in the art that other variations and/or modifications may be made to the specific embodiments without departing from the scope of the invention as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of generating treatment regimens for a medical condition for a subject in need thereof, the method comprising,
parsing pre-processed data of the subject through an optimizer to obtain a list of feasible treatment regimens;
parsing a selected subset of the list of feasible treatment regimens through a trained artificial intelligence (AI) model to obtain predicted clinical outcomes for each of the selected subset of the list of feasible treatment regimens;
obtaining an associated cost for each of the selected subset of treatment regimens;
obtaining an associated preference score for each of the selected subset of treatment regimens;
generating an associated value score for each of the selected subset of treatment regimens, said associated value score based on the predicted clinical outcomes, associated costs, and associated preference scores of the selected subset of the list of feasible treatment regimens; and
generating one or more recommended treatment regimens based on the associated value scores, each recommended treatment regimen comprising a drug mix and dose regimen.

2. The method according to claim 1, wherein generating an associated value score for each of the selected subset of treatment regimens comprises,
calculating a predicted percentage improvement in clinical outcome for the selected subset of treatment regimens,
calculating a weighted percentage reduction in associated cost, and
calculating a weighted version of the associated preference score, of the selected subset of treatment regimens.

3. The method according to claim 1- or 2, wherein generating an associated value score for each of the selected subset of treatment regimens is based on an optimization of the following value function:
$$value ( Rec ) = \frac{{H}_{c} - PRED ( P , Rec )}{{H}_{c}} + {w}_{o} \frac{COST ( {Rec}_{curr} ) - COST ( Rec )}{COST ( {Rec}_{curr} )} + {w}_{1} PREF ( P , Rec )$$
wherein,
P is a patient visit profile,
Rec_{curr} is a current treatment regimen,
Rec is a recommended treatment regimen,
H_{c} is a pre-visit measurement of the clinical outcome,
PRED is a clinical outcome prediction function of the trained AI model which outputs a predicted clinical outcome based on an input treatment regimen,
PREF is a preference scoring function which outputs a score of an input treatment regimen,
COST is a cost function which outputs a cost of an input treatment regimen,
w₀ is a hyperparameter which controls a trade-off between improvement in clinical outcome and cost,
w₁ is a hyperparameter which controls a trade-off between improvement in clinical outcome and a preferred medication prescription; and
optionally wherein generating one or more recommended treatment regimens is based on obtaining one or more treatment regimens that maximize the value function.

4. (canceled)

5. The method according to claim 3, wherein parsing the pre-processed data of the subject through an optimizer to obtain the list of feasible treatment regimens comprises accessing a clinical knowledge database to obtain the list of feasible treatment regimens.

6. The method according to claim 5, wherein parsing a selected subset of the list of feasible treatment regimens through a trained artificial intelligence (AI) model comprises,
selecting, from the list of feasible treatment regimens, a first list of feasible treatment regimens comprising a first medication;
sorting the first list of feasible treatment regimens based on a dosage quantity of the first medication to obtain a first ordered list of feasible doses of the first medication;
obtaining a first ordered list of treatment regimens corresponding with the first ordered list of feasible doses of the first medication;
parsing the first ordered list of treatment regimens to obtain an inflection treatment regimen associated with an inflection point of the value function, using a first binary search algorithm, and parsing the first ordered list of treatment regimens between the inflection treatment regimen and a maximum treatment regimen, using a second binary search algorithm, to obtain one or more recommended treatment regimens each having an associated value score that optimizes the value function;
optionally wherein parsing a selected subset of the list of feasible treatment regimens through a trained artificial intelligence (AI) model further comprises,
selecting, from the list of feasible treatment regimens, a second list of feasible treatment regimens that excludes the first medication;
sorting the second list of feasible treatment regimens based on a dosage quantity of a second medication to obtain a second ordered list of feasible doses of the second medication;
obtaining a second ordered list of treatment regimens corresponding with the second ordered list of feasible doses of the second medication;
parsing the second ordered list of treatment regimens to obtain an inflection treatment regimen associated with an inflection point of the value function, using the first binary search algorithm, and
parsing the second ordered list of treatment regimens between the inflection treatment regimen and a maximum treatment regimen of the second medication, using the second binary search algorithm, to obtain one or more recommended treatment regimens each having an associated value score that optimizes the value function;
and optionally wherein parsing a selected subset of the list of feasible treatment regimens through a trained artificial intelligence (AI) model further comprises,
selecting, from the list of feasible treatment regimens, a third list of feasible treatment regimens comprising a specific dosage of the first medication;
sorting the third list of feasible treatment regimens based on a dosage quantity of a third medication to obtain a third ordered list of feasible doses of the third medication;
obtaining a third ordered list of treatment regimens corresponding with the third ordered list of feasible doses of the third medication;
parsing the third ordered list of treatment regimens to obtain an inflection treatment regimen associated with an inflection point of the value function, using the first binary search algorithm, and
parsing the third ordered list of treatment regimens between the inflection treatment regimen and a maximum treatment regimen, using the second binary search algorithm, to obtain one or more recommended treatment regimens each having an associated value score that optimizes the value function.

7. (canceled)

8. (canceled)

9. The method according to claim 1, wherein the associated preference score for each of the selected subset of treatment regimens is based on one or more of:
indicator functions of whether a prioritized medication is prescribed;
linear functions in the dose of each medication; and
indicator functions of whether each medication is prescribed.

10. The method according to claim 1, further comprising pre-processing data of the subject by a data pre-processor, said pre-processing step comprising,
extracting one or more drug mixes and dose regimens from an electronic medical record (EMR) of the subject;
computing a first vector representing a drug mix and dose regimen of a first visit treatment regimen, wherein the first vector has a length equal to the number of generic name drugs for the medical condition, and each entry in the first vector represents a periodic dose of a corresponding generic name drug; and
computing a second vector representing a drug mix and dose regimen of a second visit treatment regimen, wherein the second vector has the same length as the first vector, and each entry in the second vector represents the periodic dose of a corresponding generic name drug at the second visit;
and optionally further comprising,
performing a plurality of validation checks on the data of the subject to be pre-processed; and
triggering an alert message when a failure in one of the validation checks is detected.

11. (canceled)

12. The method according to claim 1, further comprising training the AI model, said training comprising,
pre-processing training data comprising retrospective data from electronic medical records of a group of patients having the medical condition; the electronic medical records comprising the patients' medical profile data, historical drug mixes and dose regimen data and associated clinical outcome for each historical drug mix and dose regimen;
feeding the pre-processed training data into a machine learning algorithm to model clinical outcomes under different drug mixes and dose regimens; and
obtaining the trained AI model that is configured to predict clinical outcomes using the subject's medical profile and a treatment regimen as input.

13. The method according to claim 3, further comprising
displaying a first and second recommended treatment regimens, predicted clinical outcomes and associated costs via an interactive dashboard;
wherein the first recommended treatment regimen is generated based on an optimization of the value function with the hyperparameter w₀ set to zero; and
wherein the second recommended treatment regimen is generated based on an optimization of the value function with the hyperparameter w₀ set to a positive value;
optionally further comprising,
regenerating the second recommended treatment regimen displayed on the interactive dashboard in response to user input to generate a lower-cost option, said regenerating step comprising,
increasing the value of the hyperparameter w₀;
generating an associated value score for each of the selected subset of treatment regimens by optimizing the value function based on the increased value of the hyperparameter w₀;
generating a lower-cost treatment regimen based on the associated value score; and
presenting the lower-cost treatment regimen as the regenerated second recommended treatment regimen;
wherein the regeneration step is configured to be triggered iteratively by the user until the lowest-cost option is presented as the regenerated second recommended treatment regimen; and
and optionally further comprising,
simulating clinical outcomes of one or more customized treatment regimens based on manually entered user input, said simulation step comprising,
preprocessing the one or more customized treatment regimens;
parsing the one or more customized treatment regimens through the trained AI model to obtain predicted clinical outcomes for the one or more customized treatment regimens; and
presenting the predicted clinical outcomes for the one or more customized treatment regimens via the interactive dashboard.

14. (canceled)

15. (canceled)

16. A system for generating treatment regimens for a medical condition for a subject in need thereof, the system comprising,
an optimizer for parsing pre-processed data of the subject to obtain a list of feasible treatment regimens; and
a trained AI model for parsing a selected subset of the list of feasible treatment regimens to obtain predicted clinical outcomes for each of the selected subset of the list of feasible treatment regimens;
wherein the optimizer is further configured to:
obtain an associated cost for each of the selected subset of treatment regimens;
obtain an associated preference score for each of the selected subset of treatment regimens;
generate an associated value score for each of the selected subset of treatment regimens, said associated value score based on the predicted clinical outcomes, associated costs, and associated preference scores of the selected subset of the list of feasible treatment regimens; and
generate one or more recommended treatment regimens based on the associated value scores, each recommended treatment regimen comprising a drug mix and dose regimen.

17. The system according to claim 16, wherein the optimizer is configured to generate an associated value score for each of the selected subset of treatment regimens by calculating a predicted percentage improvement in clinical outcome for the selected subset of treatment regimens, calculating a weighted percentage reduction in associated cost, and calculating a weighted version of the associated preference score of the selected subset of treatment regimens.

18. The system according to claim 16, wherein the optimizer is configured to generate an associated value score for each of the selected subset of treatment regimens based on an optimization of the following value function:
$$value ( Rec ) = \frac{{H}_{c} - PRED ( P , Rec )}{{H}_{c}} + {w}_{o} \frac{COST ( {Rec}_{curr} ) - COST ( Rec )}{COST ( {Rec}_{curr} )} + {w}_{1} PREF ( P , Rec )$$
wherein,
P is a patient visit profile,
Rec_{curr} is a current treatment regimen,
Rec is a recommended treatment regimen,
H_{c} is a pre-visit measurement of the clinical outcome,
PRED is a clinical outcome prediction function of the trained AI model which outputs a predicted clinical outcome based on an input treatment regimen,
PREF is a preference scoring function which outputs a score of an input treatment regimen,
COST is a cost function which outputs a cost of an input treatment regimen,
w₀ is a hyperparameter which controls a trade-off between improvement in clinical outcome and cost,
w₁ is a hyperparameter which controls a trade-off between improvement in clinical outcome and a preferred medication prescription; and
optionally wherein the optimizer is configured to generate one or more recommended treatment regimens that maximize the value function.

19. (canceled)

20. The system according to claim 18, further comprising a clinical knowledge database coupled to the optimizer, wherein the optimizer is configured to access the clinical knowledge database to obtain the list of feasible treatment regimens.

21. The system according to claim 20, wherein the optimizer is configured to cooperate with the trained AI model to generate the one or more recommended treatment regimens by,
selecting, from the list of feasible treatment regimens, a first list of feasible treatment regimens comprising a first medication;
sorting the first list of feasible treatment regimens based on a dosage quantity of the first medication to obtain a first ordered list of feasible doses of the first medication;
obtaining a first ordered list of treatment regimens corresponding with the first ordered list of feasible doses of the first medication;
parsing the first ordered list of treatment regimens to obtain an inflection treatment regimen associated with an inflection point of the value function, using a first binary search algorithm, and parsing the first ordered list of treatment regimens between the inflection treatment regimen and a maximum treatment regimen, using a second binary search algorithm, to obtain one or more recommended treatment regimens each having an associated value score that optimizes the value function;
optionally wherein the optimizer is further configured to cooperate with the trained AI model to generate the one or more recommended treatment regimens by,
selecting, from the list of feasible treatment regimens, a second list of feasible treatment regimens that excludes the first medication;
sorting the second list of feasible treatment regimens based on a dosage quantity of a second medication to obtain a second ordered list of feasible doses of the second medication;
obtaining a second ordered list of treatment regimens corresponding with the second ordered list of feasible doses of the second medication;
parsing the second ordered list of treatment regimens to obtain an inflection treatment regimen associated with an inflection point of the value function, using the first binary search algorithm, and
parsing the second ordered list of treatment regimens between the inflection treatment regimen and a maximum treatment regimen of the second medication, using the second binary search algorithm, to obtain one or more recommended treatment regimens each having an associated value score that optimizes the value function;
and optionally wherein the optimizer is further configured to cooperate with the trained AI model to generate the one or more recommended treatment regimens by,
selecting, from the list of feasible treatment regimens, a third list of feasible treatment regimens comprising a specific dosage of the first medication;
sorting the third list of feasible treatment regimens based on a dosage quantity of a third medication to obtain a third ordered list of feasible doses of the third medication;
obtaining a third ordered list of treatment regimens corresponding with the third ordered list of feasible doses of the third medication;
parsing the third ordered list of treatment regimens to obtain an inflection treatment regimen associated with an inflection point of the value function, using the first binary search algorithm, and
parsing the third ordered list of treatment regimens between the inflection treatment regimen and a maximum treatment regimen, using the second binary search algorithm, to obtain one or more recommended treatment regimens each having an associated value score that optimizes the value function.

22. (canceled)

23. (canceled)

24. The system according to claim 16, wherein the associated preference score for each of the selected subset of treatment regimens is based on one or more of:
indicator functions of whether a prioritized medication is prescribed;
linear functions in the dose of each medication; and
indicator functions of whether each medication is prescribed.

25. The system according to claim 16, further comprising a data pre-processor configured to,
extract one or more drug mixes and dose regimens from an EMR of the subject;
compute a first vector representing a drug mix and dose regimen of a first visit treatment regimen, wherein the first vector has a length equal to the number of generic name drugs for the medical condition, and each entry in the first vector represents a periodic dose of a corresponding generic name drug; and
compute a second vector representing a drug mix and dose regimen of a second visit treatment regimen, wherein the second vector has the same length as the first vector, and each entry in the second vector represents the periodic dose of a corresponding generic name drug at the second visit treatment regimen;
and optionally further comprising an input validation processor configured to,
perform a plurality of validation checks on the data of the subject to be pre-processed; and
trigger an alert message when a failure in one of the validation checks is detected.

26. (canceled)

27. The system according to claim 16,
wherein the AI model is configured to predict the clinical outcome using the subject's medical profile and a treatment regimen as input; and
wherein the AI model is trained by,
pre-processing training data comprising retrospective data from electronic medical records of a group of patients having the medical condition; the electronic medical records comprising the patients' medical profile data, historical drug mixes and dose regimen data and associated clinical outcome for each historical drug mix and dose regimen; and
feeding the pre-processed training data into a machine learning algorithm to model clinical outcomes under different drug mixes and dose regimens.

28. The system according to claim 18, further comprising an interactive dashboard configured to,
display a first and second recommended treatment regimens, predicted clinical outcomes and associated costs;
wherein the first recommended treatment regimen is generated based on an optimization of the value function with the hyperparameter w₀ set to zero; and
wherein the second recommended treatment regimen is generated based on an optimization of the value function with the hyperparameter w₀ set to a positive value;
optionally wherein the optimizer is further configured to regenerate the second recommended treatment regimen in response to user input to generate a lower-cost option for display on the interactive dashboard; and
wherein the optimizer is configured to iteratively regenerate the second recommended treatment regimen in response to user input such that with each iteration, a lower-cost option is generated for display on the interactive dashboard until the lowest-cost option is presented as the regenerated second recommended treatment regimen.

29. (canceled)

30. The system according to claim 28, further configured to simulate clinical outcomes of one or more customized treatment regimens based on manually entered user input;
wherein the data pre-processor is configured to preprocess the one or more customized treatment regimens;
the trained AI model is configured to parse the one or more customized treatment regimens to obtain predicted clinical outcomes for the one or more customized treatment regimens; and
the interactive dashboard is configured to present the predicted clinical outcomes for the one or more customized treatment regimens.

31. A non-transitory computer readable storage medium having stored thereon instructions for instructing a processing unit of an apparatus to execute a method of generating treatment regimens for a medical condition for a subject in need thereof, the method comprising,
parsing pre-processed data of the subject through an optimizer to obtain a list of feasible treatment regimens;
parsing a selected subset of the list of feasible treatment regimens through a trained artificial intelligence (AI) model to obtain predicted clinical outcomes for each of the selected subset of the list of feasible treatment regimens;
obtaining an associated cost for each of the selected subset treatment regimens;
obtaining an associated preference score for each of the selected subset of treatment regimens;
generating an associated value score for each of the selected subset of treatment regimens, said associated value score based on the predicted clinical outcomes, associated costs, and associated preference score of the selected subset of the list of feasible treatment regimens; and
generating one or more recommended treatment regimens based on the associated value scores, each recommended treatment regimen comprising a drug mix and dose regimen.
